(19) United States
(12) Patent Application Publication
(10) Pub. No.: US 2023/0414972 A1
(43) Pub. Date: Dec. 28, 2023

(54) SYSTEMS AND METHODS OF ALIGNMENT CONTROL FOR NEUROMODULATION DELIVERY SYSTEM

(71) Applicant: General Electric Company, Schenectady, NY (US)

(72) Inventors: David Andrew Shoudy, Niskayuna, NY (US); Weston Blaine Griffin, Niskayuna, NY (US); Radhika Madhavan, Niskayuna, NY (US); Jeffrey Michael Ashe, Gloversville, NY (US)

(21) Appl. No.: 17/809,311

(22) Filed: Jun. 28, 2022

(51) Int. Cl. A61N 7/00; A61B 8/00; A61B 8/08

(52) U.S. Cl. CPC A61N 7/00; A61B 8/4245; A61B 8/54; A61B 8/085; A61N 2007/0026; A61N 2007/0095

(57) ABSTRACT

The present discussion relates to structures and devices to facilitate application of an ultrasound therapy beam to a target anatomic region in a replicable manner. In certain aspects, an alignment controller may be used to analyze images generated by an ultrasound transducer. The alignment controller may then send a communication to indicate the energy application device is positioned to provide therapy to the target region, or if the device needs to be repositioned. The alignment control of the energy application device provides guided repeatable targeting of the target anatomic region, even when in non-clinical settings.

### BACKGROUND

The subject matter disclosed herein relates to an alignment control system for targeting and/or dosing regions of interest in a human subject via application of neuromodulating energy to cause targeted physiological outcomes. Specifically, a system for positioning and orienting an energy application device in a way that an untrained person may provide treatments to an anatomic target for human subject treatment.

Neuromodulation has been used to treat a variety of clinical conditions. However, specific tissue targeting via neuromodulation may be challenging. For example, accurate focusing of neuromodulating energy may vary based on individual human subject anatomy. Certain subjects may have variations in organ size or location relative to other subjects based on their height, weight, age, gender, clinical condition, and so forth, which may impact targeting when using various neuromodulation techniques.

In the context of neuromodulation using ultrasonic devices, other common challenges may relate to the difficulty in repeatedly delivering accurate and consistent ultrasonic therapy at a prescribed dose in the context of a treatment regime involving multiple, repeated treatments of the treatment region. Further, such treatments may be difficult for a minimally trained person to administer, making it necessary for the human subject to enter a clinical setting and/or be treated by medically trained personnel for each treatment session. Treatment by the human subject themselves, or in a home setting, is therefore not typically considered feasible for an ultrasound-based neuromodulation regime.

For example, when a clinician administers a conventional ultrasound exam, the clinician places the probe on the body and maneuvers in all degrees-of-freedom (DOFs) until they arrive at the target scan plane. In contrast, in a human subject or self-administered, at-home context, the untrained user has little to no capacity to understand an ultrasound image, if available, and intelligently maneuver a handheld ultrasound probe to find the target. Such issues make self-administration of precisely targeted ultrasonic based treatments, particularly in an at-home context, impractical using conventional approaches.

### BRIEF DESCRIPTION

The disclosed embodiments are not intended to limit the scope of the claimed subject matter, but rather these embodiments are intended only to provide a brief summary of possible embodiments. Indeed, the disclosure may encompass a variety of forms that may be similar to or different from the embodiments set forth below.

In one embodiment, a neuromodulation delivery system includes an energy application device and an alignment controller. The alignment controller is configured to perform acts including receiving image data from the energy application device, wherein the image data comprises images of internal tissue based on a current position and orientation of the energy application device relative to a subject, and determining an alignment score of the energy application device with respect to an anatomic target based on the image data. Further, the alignment controller is configured to provide a control signal to maintain or change one or both of the current position or orientation of the energy application device in response to the alignment score.

In another embodiment, a method includes receiving, via a processor, time-series image data from an energy application device at a current position and orientation relative to a subject, wherein the time-series image data comprises images of internal tissue of the subject at the current position and orientation over time. Additionally the method includes determining, via the processor, an alignment score of the energy application device relative to an anatomic target over time based on the image data, and comparing, via the processor, the alignment score over a time interval corresponding to the time-series image data at the current position and orientation to a predicted alignment score for the time interval at one or both of an additional position or orientation. Further the method includes providing a control signal to maintain or change one or both of position or orientation of the energy application device based on the comparison.

In a further embodiment, a tracking system includes one or more processors and a memory, wherein the one or more processors are configured to execute instructions stored on the memory to perform acts comprising receiving image data from an energy application device, wherein the image data comprises images of internal tissue based on a current position and orientation of the energy application device relative to a subject. The acts further include receiving target image data comprising an anatomic target and corresponding to internal tissue of the subject, identifying the anatomic target in an image frame of the image data and tracking the anatomic target in subsequent frames of the image data, and determining an alignment score of the energy application device relative to the anatomic target based on the image data. Additionally, the acts include comparing the alignment score to a threshold relative to the anatomic target, and providing a control signal to maintain or change one or both of position or orientation of the energy application device based on the comparison of the alignment score to the threshold.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 a block diagram of a neuromodulation delivery system, in accordance with embodiments of the present disclosure;

FIG. 2 is a schematic diagram of a probe of the neuromodulation delivery system of FIG. 1, in accordance with embodiments of the present disclosure;

FIG. 3 is a flow diagram of a method of alignment control of the probe of the neuromodulation delivery system of FIG. 1, in accordance with embodiments of the present disclosure;

FIG. 4 is an illustration of a user interface for alignment control of the probe of the neuromodulation delivery system of FIG. 1, in accordance with embodiments of the present disclosure;

FIG. 5 is a flow diagram of a method of delivering a therapy dose via the probe of the neuromodulation delivery system of FIG. 1, in accordance with embodiments of the present disclosure;

FIG. 6 is an illustration of a user interface for therapy dosing via the probe of the neuromodulation delivery system of FIG. 1, in accordance with embodiments of the present disclosure;

FIG. 7 is a schematic diagram of tracking over time using a therapy transducer that has adjustable focus in one-dimension, in accordance with embodiments of the present disclosure;

FIG. 8 is a timing diagram of portal-vein motion within ultrasound image data and inertial measurement unit (IMU) sensor data, in accordance with embodiments of the present disclosure;

FIG. 9A is a schematic diagram of tracking over time using a fixed two-dimensional imaging transducer with a mechanically rocked therapy transducer with external motion adjustment, in accordance with embodiments of the present disclosure;

FIG. 9B is a schematic diagram of tracking over time using a fixed two-dimensional imaging transducer with a mechanically rocked therapy transducer with internal motion adjustment, in accordance with embodiments of the present disclosure;

FIG. 10A is a schematic diagram of tracking over time using a two-dimensional imaging transducer coupled to the therapy transducer with external motion adjustment, in accordance with an embodiment of the present disclosure;

FIG. 10B is a schematic diagram of tracking over time using a two-dimensional imaging transducer coupled to the therapy transducer with internal motion adjustment, in accordance with an embodiment of the present disclosure;

FIG. 11 is a schematic diagram of tracking over time using a single linear transducer used for imaging and therapy, in accordance with an embodiment of the present disclosure;

FIG. 12A is a schematic diagram of three-dimensional tracking from an azimuth view with active one-dimensional focus, in accordance with an embodiment of the present disclosure;

FIG. 12B is a schematic diagram of three-dimensional tracking from an elevation view with active one-dimensional focus, in accordance with an embodiment of the present disclosure;

FIG. 13 is an illustration of a transducer face that includes a two-dimensional imaging array and a two-dimensional therapy array, in accordance with an embodiment of the present disclosure; and

FIG. 14 is a schematic diagram of an example of a fixed arrangement of a two-dimensional dual transducer for imaging and therapy, in accordance with an embodiment of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Any examples or illustrations given herein are not to be regarded in any way as restrictions on, limits to, or express definitions of, any term or terms with which they are utilized. Instead, these examples or illustrations are to be regarded as being described with respect to various particular embodiments and as illustrative only. Those of ordinary skill in the art will appreciate that any term or terms with which these examples or illustrations are utilized will encompass other embodiments that may or may not be given therewith or elsewhere in the specification and all such embodiments are intended to be included within the scope of that term or terms. Language designating such non-limiting examples and illustrations includes, but is not limited to: “for example,” “for instance,” “such as,” “e.g.,” “including,” “in certain embodiments,” “in some embodiments,” and “in one (an) embodiment.”

As discussed herein, one issue that can arise with respect to therapy techniques that involve multiple sessions (e.g., 1× per day, 3× per week, 1× per week) of targeted ultrasonic neuromodulation is the need to provide consistent, correct alignment for each session. In the context of a therapy capable of being implemented in non-clinical settings (e.g., at home) by individuals with little or no medical training (including human subjects) it is desirable that such targeting and alignment guidance be provided in as simple a format as possible. For example, it may be desirable to provide targeting and/or alignment assistance without manual guidance based on displayed images and/or with common sources of user error or frustration removed or minimized. In addition, it may further be useful to use one type of alignment device that is suitable for use across a wide subject population, despite the fact that a “one size fits all approach” is not feasible due to the large variation in human subject internal and external anatomy. With this in mind the presently described approaches, structures, and techniques encompass ultrasound therapy alignment devices having customizable elements that direct the user and/or auto-position the therapy device. The systems and devices are customizable to address the large variability in the overall human subject population, while also being suitable for use in non-clinical settings (e.g., home use by the human subject).

The human subject may position the therapy device to target an anatomic target (e.g., anatomic target for therapy treatment) within the human subject tissue, the therapy device may include alignment control to enable placing the therapy device at a position and orientation that provides effective therapy to the anatomic target. To address the uniqueness of each human subject's internal anatomy, the alignment device may include angular and/or orientation adjustments (e.g., rock, tilt, spin) and configurable depth to nominally point and focus the therapy beam at the internal anatomic target region. Further, in some embodiments, after the location of the anatomic target within one or more image frames is determined, the system may deliver therapy at that location by electronically steering the therapy beam to the anatomic target when applying a therapy dose. Alternatively, a therapy transducer having appropriate therapy transducer characteristics (e.g., power handling, frequency range, geometry and so forth) and/or a probe cap having a suitable probe cap characteristics (e.g., angular adjustment, attenuation adjustment (for example, standoff height and/or composition), other geometries or features useful for focusing, shaping, or targeting the beam, and so forth) may be selected and employed to direct the therapy beam to the determined location when applying a therapy dose.

In further examples, certain implementations discussed herein utilize an alignment controller of the system to enable a user to position the energy application device so as to direct the energy application device toward a region in which an anatomic target (e.g., clinician predetermined anatomic target) of the human subject is located. The alignment controller may receive sensor data from multiple sensors that may monitor a human subject's movement throughout a respiration cycle and/or collect image frame data of the human subject's internal tissue. The alignment controller may send a signal to one or more components of the energy application device to steer and/or focus the therapy beam so as to correspond to the human subject's movement relative to the anatomic target predefined for the human subject. This may enable the therapy dose to be delivered accurately during the subject's body surface movement and internal movement caused by the respiration cycle of the subject. The alignment controller may utilize image data over time to send one or more signals to adjust the position (e.g., two-dimensional plane, three-dimensional position) and orientation (e.g., rock, tilt, spin) of the therapy beam of the energy application device over time to correspond to the position of the anatomic target within the subject. The alignment may be manually accomplished, such as through alignment signals that may be displayed on a display of the device or a subject electronic device for the subject to follow to reposition the energy application device. Additionally, the alignment may be automatic such that the alignment controller may send signals to electronically steer and/or focus the energy application device to the desired position and/or orientation. The automation controller, upon receipt of the signal, may automatically adjust the probe module position relative to the user (e.g., on or external to the user), adjust an internal position of the probe transducers within the probe housing, or both to improve alignment to the anatomic target. The automation controller may perform internal and/or external adjustments of the probe module until the alignment meets a target and/or threshold alignment.

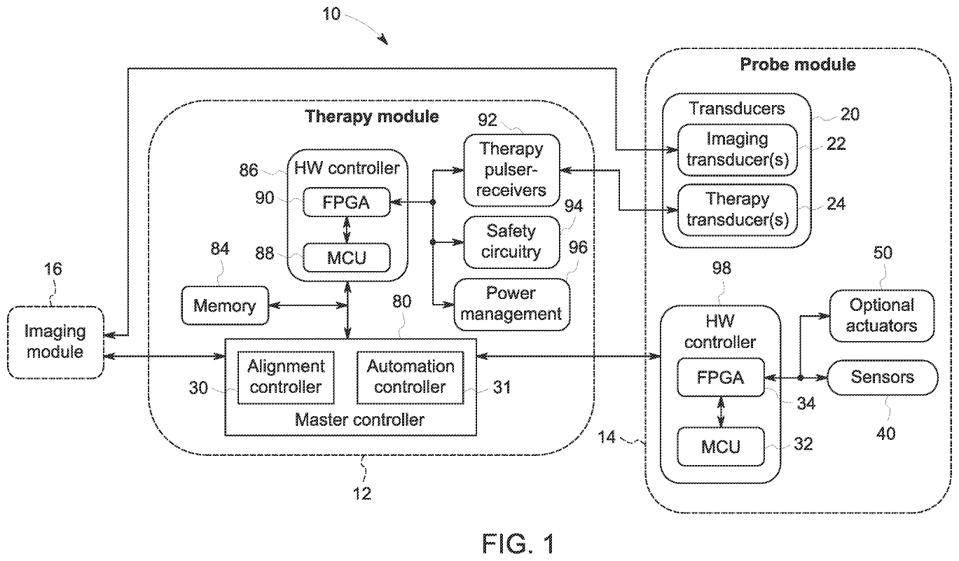

With this in mind, FIG. 1 depicts an example of a neuromodulation system configured to be used to deliver neuromodulating energy as part of a treatment protocol and which may be used with an alignment and/or positioning device or structure as discussed herein. In particular, FIG. 1 is a schematic representation of a system 10 for neuromodulation to achieve neuromodulating effects such as neurotransmitter release and/or activation of components (e.g., the presynaptic cell, the postsynaptic cell) of a synapse in response to an application of energy. The system 10 includes a pulse generator (as part of a therapy module 12) coupled to an energy application device (one or more therapy transducers 24 depicted as part of the probe module 14). The energy application device is configured to receive, e.g., via leads or wireless connection, or otherwise generate energy pulses that in use are directed to an anatomic target of a subject, which in turn results in a targeted physiological outcome.

In certain embodiments, the energy application device and/or the pulse generator may communicate wirelessly, for example with a controller that may in turn provide instructions to the pulse generator. As discussed herein, the energy application device may be an extracorporeal device, e.g., may operate to apply energy transdermally or in a noninvasive manner from a position outside of a subject's body, and may, in certain embodiments, be integrated with the pulse generator and/or the controller. In embodiments in which the energy application device is extracorporeal, the energy application device may be operated by a caregiver, or the subject, and positioned at a spot on or above a subject's skin such that the energy pulses are delivered transdermally to a desired internal tissue. Once positioned to apply energy pulses to a region of interest, the system 10 may initiate neuromodulation of one or more nerve pathways to achieve a targeted physiological outcome or clinical effect. In some embodiments, the system 10 may be implemented such that some or all of the elements may communicate in a wired or wireless manner with one another.

The system 10 may include an alignment controller 30 that assesses characteristics that are indicative of the placement and orientation of the energy application device. Based on such an assessment, delivery of therapeutic ultrasonic energy may be altered, modulated, or steered automatically and/or manually to achieve the prescribed therapeutic result. By way of example, the therapy beam may be electronically steered to the target location when applying a therapy dose. In addition or in the alternative, an indication or guidance may be provided to the user, such as via audible, visible, or haptic indicators, to provide guidance regarding placement and/or orientation of the energy application device.

The energy application device as provided herein may provide energy pulses according to various modulation parameters as part of a treatment protocol to apply a prescribed amount of energy. For example, the modulation parameters may include various stimulation time patterns, ranging from continuous to intermittent. With intermittent stimulation, energy is delivered for a period of time at a certain frequency during a signal-on time. The signal-on time is followed by a period of time with no energy delivery, referred to as signal-off time. The modulation parameters may also include frequency and duration of a stimulation application. The application frequency may be continuous or delivered at various time periods, for example, within a day or week. Further, the treatment protocol may specify a time of day to apply energy or a time relative to eating or other activity. The treatment duration to cause the targeted physiological outcomes may last for various time periods, including, but not limited to, from a few minutes to several hours. In certain embodiments, treatment duration with a specified stimulation pattern may last for one hour, repeated at, intervals, e.g., 72 hour intervals. In certain embodiments, energy may be delivered at a higher frequency, say every three hours, for shorter durations, for example, 30 minutes. The application of energy, in accordance with modulation parameters, such as the treatment duration, frequency, and amplitude, may be adjustably controlled to achieve a desired physiological or therapeutic result.

With the preceding context in mind, additional features illustrated in FIG. 1 are described in greater detail. In particular, aspects and components of an implementation of the system 10 are shown corresponding to certain functionalities described above. As noted above, the block diagram of FIG. 1 illustrates a therapy module 12 and a probe module 14 which may be used to perform therapeutic functions described herein. An imaging module 16 is also illustrated, though it may be appreciated that in certain embodiments such an imaging module 16 may be absent. In such alternative embodiments analytics performed on imaging data may be performed on unreconstructed (i.e., raw) imaging data or on image data that is reconstructed but not displayed. By way of example, therapy administration and/or control based on data acquired using imaging transducers or at the imaging module 16 may be based on reconstructed images (e.g., signatures within the reconstructed image data) or based on ultrasound signatures present in the unreconstructed image data.

Beginning with the probe module 14, in the depicted example the probe module 14 includes transducers 20. As used herein, a “transducer” refers to any arbitrarily sized and segmented physical structure for converting to and/or from a first energy source (i.e., electrical, mechanical, magnetic, etc.) and ultrasonic energy, where the probe module 14 includes a collection of one or more transducers. As discussed herein, the geometry of the collection of transducers could be a linear (1D) array, an area (2D) array, or any other suitable geometry of any size, while the imaging and therapy transducers as described herein could be independent (i.e., separate and distinct structures), partially shared (i.e., having partial overlap between the structures used to generate the therapy and imaging ultrasonic energy), or fully shared (i.e., complete overlap between the structures used to generate the therapy and imaging ultrasonic energy). In some contexts, the “imaging transducer” or “therapy transducer” may be used to refer to the collection of one or more transducers used for the associated imaging or therapy function. In other contexts, an “imaging beam” or “therapy beam” may be discussed which is generated from a collection of one or more transducers, wherein the collection of transducers used for generating the imaging and therapy beams may be independent, partially shared, or fully shared.

With this in mind, in the depicted example, the transducers 20 include both imaging transducers 22 and therapy transducers 24. In one implementation, the therapy transducer(s) 24 may operate at a frequency within 0.2 MHz to 2 MHz (such as 0.5 MHz or 2 MHz). The probe module 14 and/or transducer(s) 20 may be selectable or swappable in certain implementations to allow a clinician to choose an appropriate probe module model or type to best fit the subject or target region context, such as by allowing the clinician to select the probe module 14 and/or transducer(s) 20 having suitable nominal depth, axial focus location characteristics, power handling, frequency range, angular adjustment, attenuation adjustment, and so forth. Further, in multi-transducer embodiments, a clinician may customize the probe module 14 by selecting the subset of transducers for activation to enable coherent summation of a therapy beam at a target region of anatomy with minimal interference by obstructing anatomic structures (e.g., ribs, and so forth).

In alternative embodiments, transducers 20 may instead comprise one type of transducer capable of operating at both the respective imaging and therapy frequencies (e.g., 0.2 MHz to 2 MHz during therapy operation and 2 MHz to 12 MHz during imaging operation) such that separate transducers are not provided for each type of respective operation. In such embodiments the single transducer or type of transducer may be operated to both provide therapy and acquire imaging-type data. Such single transducer type approaches may be suitable in contexts where the target region is shallow and/or high power is not necessitated. The probe module 14 and/or transducer(s) 20 may be selectable or swappable in certain implementations to allow a clinician to choose an appropriate probe module model or type to best fit the subject or target region context, such as by allowing the clinician to select the probe module 14 and/or transducer(s) 20 having suitable nominal depth, axial focus location characteristics, power handling, frequency range, angular adjustment, attenuation adjustment, and so forth.

In the depicted example the probe module 14 includes a microcontroller(s) (MCU) 32 in communication with a master controller (e.g., processor) 80 of the therapy module 12 and a field-programmable gate array (FPGA) 34 in communication with the MCU 32 and sensors 40 and/or actuators 50 that may be present and associated with the probe module 14. In this configuration the MCU 32 and FPGA 34 may bi-directionally communicate with components of the master controller 80 to coordinate and/or record operation of aspects of the probe module 14 or, if present, components associated directly or indirectly with the probe module 14, such as actuators 50 and/or sensors 40. With respect to the sensors 40, various types of sensors may be integrated with or, if separate, in communication with the probe module 14. By way of example the sensors 40 may include one or more of inertial measurement units (IMUs) (which may function as posture sensors) the IMUs may include one or more of accelerometers, gyroscopes, and magnetometers. As shown in FIG. 1, the one or more sensors 40, if present, may be communicatively coupled to the FPGA 34 or otherwise to the hardware controller 98. In some embodiments, the master controller 80 may include an alignment controller 30 that determines an alignment score based on an orientation and position of the energy application device and ability to apply therapy to the anatomic target or region at the orientation and position. In other embodiments, the alignment of the energy application device may be determined via a software-implemented, firmware-implemented, and/or hardware-implemented function of the alignment controller 30. Additionally, the alignment controller 30 may communicate with an automation controller 31 that may automatically adjust orientation and/or position of the probe 14, the therapy transducer 24, or any other element of the energy application device. It should be understood, that although the alignment controller 30 and the automation controller 31 are depicted as within the master controller 80, the alignment controller 30 and/or the automation controller 31 may be located in any suitable location within the energy application device (i.e., they may be implemented as separate and distinct controllers in certain embodiments). Additionally, the alignment controller 30 may include any suitable hardware, firmware, and/or software to determine alignment of the energy application device. The one or more controllers (e.g., alignment controller 30, automation controller 31) depicted within the energy application device may be software modules and/or routines running or implemented by dedicated circuitry on the master controller 80, or could be separated out into various hardware blocks (e.g., with additional processing units).

Regarding the therapy module 12, as previously noted, implementations of the therapy module 12 may include a master controller (e.g., processor) 80 which may itself include or execute various sub-modules or routines, such as may be stored on a memory structure 84. For example, the master controller 80 may include or execute modules or routines providing functionality for image streaming and remote control, alignment control, artificial intelligence (AI) anatomy recognition and tracking, dose accumulation, user interface, support analytics, system guidance and automation, a data logger and so forth.

As with the probe module 14, in certain embodiments the therapy module 12 may include a hardware controller 86 which may include its own MCU 88 and FPGA 90. While depicted as separate modules for the purpose of illustration and explanation, in practice the probe module 14 and therapy module 12 may actually be one and the same (i.e., an integral structure or device configured to perform the functions of both the therapy module and probe module as discussed herein). With this in mind, though discussed separately herein, in practice the hardware controllers 86 and 98 may be implemented as a single hardware controller. In the depicted example the MCU 88 is depicted as being in communication with the master controller 80 and its components and modules. The FPGA 90 communicates with and/or controls other components of the therapy module 12, such as therapy pulser-receivers 92 (depicted as being in communication with the therapy transducers 24 of the probe module 14), safety circuitry 94, and/or power management circuitry 96. In practice, the master controller 80 in conjunction with the hardware controllers 86, 98 may control operation of the therapy module 12 and probe module 14, such as to perform application of therapy in accordance with processes and structures described herein. In some embodiments, the hardware controllers 86, 98 may not have the MCU 88 and/or FPGA 90. Additionally, the system 10 may include a single hardware controller and/or multiple hardware controllers. That is, the respective functionalities discussed herein as corresponding to the various controllers may be combined to a single controller, split between multiple controllers, or otherwise associated with any number of controllers or arrangements of controllers without changing the respective functionalities discussed herein. That is, the present discussion and example is intended to be illustrative and to facilitate explanation, but is not intended to limit the discussed functionalities to the described components or arrangement of components. Further, any programmable logic device and/or processor may be included in the hardware controllers 86, 98 and/or the hardware controllers 86, 98 and, as noted above, the master controller 80 may be combined into a single master processor device. In addition, as shown in FIG. 1, one or both of the master controller 80 and/or hardware controller 86 may be in communication with one or more memory structures 84 (e.g., a volatile or non-volatile memory, a firmware construct, a mass data storage, and so forth). As may be appreciated, code or executable routines for performing operations (e.g., therapy procedures or protocols) may be stored on the memory 84 for use by other components. In addition, one or more configurable parameters (e.g., system settings, imager settings, sensor settings or thresholds, and so forth) may be stored in the memory structure 84, such as by a user who has configured or calibrated the system 10 for use by a given subject for a respective therapy protocol. In addition, the memory structure 84 may be used to store data (e.g., image data) acquired or generated as part of a therapy procedure, such as for later readout and evaluation.

In the depicted example, an imaging module 16 is also depicted as being a component of the overall system. Such a module, if present, may control or monitor operation of transducers 20 (e.g., imaging transducers 22) to control generation, collection, and/or processing (e.g., reconstruction) of imaging data. In the depicted example, the imaging module 16 is also shown as being in communication with the master controller 80 of the therapy module 12, which may control operation of or respond to feedback and data from the imaging module 16. As with the probe module 14 and therapy module 12 discussed above, the imaging module 16 is illustrated as a separate module in FIG. 1 to facilitate illustration and explanation of the functional concepts. However, as with the preceding examples, the imaging module 16 may actually be one and the same with one or both of the probe module 14 and therapy module 12 (i.e., an integral structure or device configured to perform the combined functions of the imaging module and one or both of the therapy module and probe module as discussed herein).

With the preceding system description in mind and as context, the present techniques relate to an image (or unreconstructed image data) guided ultrasonic therapy system. In certain embodiments, an alignment controller 30 within the system 10 may provide targeting and/or alignment assistance used in conjunction with the energy application device discussed with respect to FIG. 1.

A variety of example embodiments of tracking and alignment methods, referred to herein collectively as tracking devices or structures, are described herein. These tracking devices may, when used, facilitate the application of safe and effective ultrasound neuromodulation therapy, including in non-clinical settings and/or when operated by untrained users (e.g., the subject themselves or other individuals lacking clinical or medical training). As such certain of the presently described embodiments are designed or configured for ease of use by the end user.

By way of context, in conventional ultrasound scans there is a high-degree of variability across the subject population. A trained sonographer adapts to such subject variability by adjusting the probe placement location on the body, probe angles, and system settings to arrive at a diagnostic image of the target. As noted herein, the present tracking methods help avoid such manual operations (thereby facilitating placement and use by an untrained individual) to allow personalized, easy, tracking of the subject region of interest and hands-free operation during a therapy session. The energy application device may be attached using a body worn fixture to the subject or may be maneuvered freely around the subject body. In other embodiments, the energy application device may initially be freely maneuvered and/or position and orientation of the device adjusted, and may be locked down at a later time using the body worn fixture.

As discussed in the previous section, the energy application device may be automatically steered and/or manually steered to the anatomic target for treatment of the subject. The therapy module 12 of the system 10 may include the alignment controller 30 that may be able to analyze the current therapy zone (e.g., region within ultrasound image data the therapy transducer 24 is able to deliver therapy to of the energy application device based on the subject ultrasound image data and assess the alignment of the current therapy zone of the probe module 14 relative to the anatomic target for subject therapy. Based on such an assessment, delivery of therapeutic ultrasonic energy may be altered, modulated, or steered automatically and/or manually to achieve the prescribed therapeutic result. By way of example, the therapy beam may be electronically steered and/or manually steered to the anatomic target when applying a therapy dose.

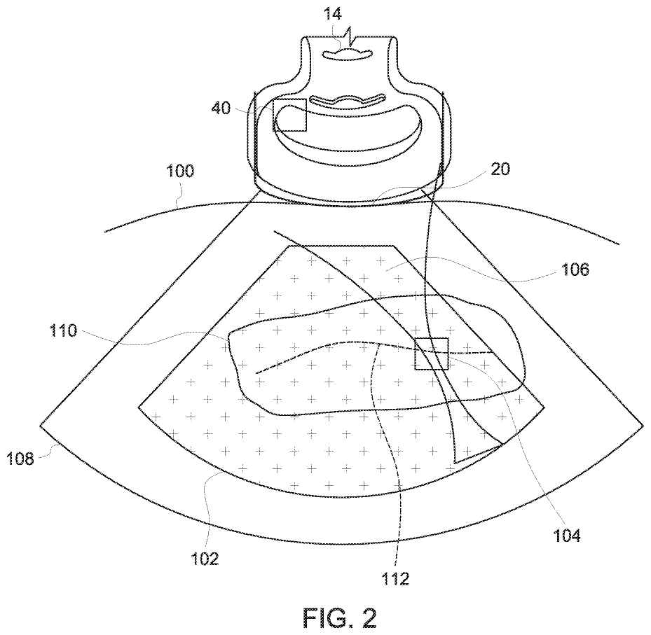

With the foregoing in mind, FIG. 2 is a schematic diagram of the probe module 14 of the system 10, in accordance with embodiments of the current disclosure. The probe 14 (e.g., energy application device) may include one or more transducers 20 (e.g., therapy transducer 22, an imaging transducer 24) and one or more position sensors 40 (e.g., inertial measurement unit (IMU) sensors, optical tracker sensors). The ultrasound probe module 14 may be placed along the subject body 100 and may include the transducers 20 that generate one or more images of internal tissue of the subject body 100 corresponding to the anatomic target 104 (e.g., organ, tissue) for therapy treatment. The probe module 14 may be automatically steered and/or manually steered (e.g., in real-time) based on image tracking and analysis via the alignment controller 30 based on subject ultrasound images and the determined therapy zone 102 (e.g., region within the ultrasound image data the therapy transducer is able to deliver therapy to) relative to the desired anatomic target 104 for the subject.

Initially, the alignment controller 30 may receive ultrasound image data (e.g., static image data and/or time-series image data corresponding to a respiration cycle of the subject). Additionally, the alignment controller 30 may receive additional data including the sensor data 40 (e.g., inertial measurement unit (IMU) data, optical tracker data). The image data received by the alignment controller 30 may include two-dimensional plane image data, three-dimensional volume image data, A-line image data, doppler image data, or other ultrasound-based image data. The alignment controller 30 may receive initial subject data (e.g., image data) relating to the anatomic target 104 for a therapy dose. The initial subject data may be determined based on past subject data, population data, or the like. The initial subject data may include ultrasound images and/or other image data of the subject's anatomy and tissue, or population-based image data related to the desired anatomic target 104. The initial subject data may include a clinician identified anatomic target 104 for therapy dosing and/or other subject image data. Additionally, the anatomic target 104 may be determined based on an AI model trained using the population data, subject specific data, or both. The AI model may be anatomy-specific, and multiple models may correspond to different anatomy regions. The subject specific AI model may learn the observed trajectory of the subject anatomic target 104, or leverage time-series images to feed forward one or more prior detections of the anatomic target 104 as an additional input for localizing the anatomic target 104 in the image frames. The AI model may also track overall cumulative and/or average alignment metrics during a dosing session and use the metrics to feed into a continuous learning algorithm that optimizes the device for the subject to maximize subject metrics over time.

The probe module 14 may be moved relative to the subject body 100 and the alignment controller 30 may receive ultrasound image data via the imaging transducer 24 of the probe module 14. The imaging transducer 24 may generate image data corresponding to a two-dimensional image plane 108, remain in unreconstructed form, or any other suitable image format. The alignment controller 30 may localize the anatomic target 104 within the received ultrasound image data by comparing the image data to previously acquired image data of the anatomic target 104 and/or previously acquired image data of the anatomical region surrounding the anatomic target 104, wherein the previously acquired image data could be subject-specific, population based, or both. The alignment controller 30 may utilize the image data to extract the location of the anatomic target 104 within the ultrasound image data, and may localize the image data relative to the anatomic target 104. It should be understood that the ultrasound image data received by the alignment controller may be fully, partially, or non-inclusive of the anatomic target 104, so in some embodiments the alignment controller 30 may not be able to localize the anatomic target 104 within the image data. The alignment controller 30 may also localize the center point within the anatomic target 104 and/or the volume of the subject that corresponds to the anatomic target 104.

Based on the received image data, the alignment controller 30 may determine an alignment score (e.g., percent alignment score, average percent alignment score, yes and/or no indication of alignment) indicating a level of alignment and/or alignment of the therapy zone 102 with the localized anatomic target 104. The alignment score may be calculated based on determining the therapy zone of the probe module 14 (e.g., region within the ultrasound image data the therapy transducer is able to deliver therapy to) and determining if the anatomic target 104 is within suitable range of the therapy zone 102. The alignment score may also be calculated based on if the anatomic target 104 is within a contour region 110 (e.g., clinician defined subject specific region that is associated with the anatomic target location) to verify reliable alignment and prevent false positives based on inaccurate anatomic target 104 detection by the alignment controller 30. It should be understood, that any suitable criteria may be used to calculate an alignment score using the alignment controller 30 based on the received image data, past subject data, population-based data, and/or clinician defined contour regions 110 within the subject image data.

In some embodiments, an alignment score may be determined, as discussed above, by the alignment controller 30 for each ultrasound image frame taken over a certain period of time (e.g., time series images). Additionally, a cumulative alignment score may be determined based on determining an average and/or weighted average of each image frame alignment score within a set of time series image data. The cumulative alignment score may be calculated by averaging the alignment scores of each image frame for image data collected within a certain time period (e.g., 5 seconds, 10 seconds 15 seconds) and/or determining an average alignment score based on one or more alignment scores associated with image frames collected over a respiration cycle of the subject.

The alignment controller 30 may analyze the image data (e.g., time series image data) and determine whether an alignment score determined for the energy application device is within a predetermined threshold range (e.g., percent alignment range) of the anatomic target 104. The alignment score may correspond to an instantaneous score based on the overlap percentage of the identified anatomic target location within the image frame data to the therapy zone 102 and/or the contour region 110 in the image frame, an accumulated score based on the percent alignment (e.g., based on each image being aligned/not aligned in each image frame, based on percent overlap of the anatomic target to therapy zone and/or contour region in each frame) over time in the image series frame data, or a combination of both methods. The predetermined threshold range may correspond to a set threshold for effective therapy dosage based on subject data. For example, an alignment score corresponding to a predetermined threshold range of 90%-100% alignment may correspond to therapy dosage application for five minutes while the alignment score is within the predetermined threshold range of 90%-100% alignment, an alignment score corresponding to a predetermined threshold range of 50%-80% alignment may correspond to therapy dosage application of 10 minutes, while the alignment score is within the predetermined threshold range of 50%-80% alignment. The therapy dosage time may be dynamically adjusted based on the alignment score threshold range, so that therapy dosage application time corresponds to, or is otherwise based on, the alignment score over time. It should be understood that any suitable therapy dosage time and/or dosing level may be applied to the subject based on the predetermined threshold range of the alignment score. Additionally, the predetermined threshold range may be any suitable range based on a fully or partially predicted dose session time, a predicted therapy efficacy based on the alignment score, or both. In some the alignment controller 30 may determine if the current alignment score is greater than one or more predicted alignment scores, and send a control signal for the energy application device to maintain the current position and orientation in the case that the current alignment score is greater than one or more predicted alignment scores. This may enable evaluation of potential positions and orientations of the energy application device relative to the current position of the energy application device in terms of dose effectiveness. A predicted alignment score may be calculated based on one or more potential positions and orientations of the energy application device and compared to the alignment score determined for the current position and orientation of the energy application device. If the alignment score for the current position and orientation of the energy application device is greater than the one or more predicted alignment scores, the energy application device may maintain current position and orientation. If a predicted alignment score is higher than the current alignment score the alignment controller 30 may send a communication to reposition the energy application device to the predicted orientation and position corresponding to the predicted alignment score. The communication may be a control signal to display an indication to the user to move or otherwise adjust the energy application device to enable manual position and orientation adjustment by the user, a control signal to the automation controller 31 to automatically adjust the energy application device position or orientation by electronically steering and/or actuating one or more therapy transducers 24 and/or the probe module 14 of the energy application device, or a combination of both manual and automatic adjustment methods.

Additionally, the alignment controller 30 may receive time-series image data and may determine if the anatomic target 104 location within the time-series image data is aligned with the energy application device's therapy zone 102 to achieve a desired percent on target dosage time to ensure an entire therapy dose may be provided within a threshold time limit (e.g., time limit determined for effective therapy dose). This time-series data may represent a target path 112 of the anatomic target 104 over time based on subject body movement (e.g., respiration cycle).

Further, the alignment controller 30 may determine an acceptable relative alignment based on the collected time-series image data and position data based on an observed trajectory of the anatomic target 104 compared to the therapy zone 102 (e.g., determined by the mechanical and/or electrical steering and/or focusing ability of the therapy beam 106 produced by the therapy transducer 22). The alignment controller 30 may determine the observed trajectory of the target path 112 of the anatomic target 104 relative to the subject body 100 during the subject respiration cycle, and the alignment controller 30 may determine a percent of on-target alignment based on the observed trajectory. In some embodiments, a learned trajectory may be stored in the memory 84 as a subject-specific trajectory, and may be used as an input for algorithms used to align the anatomic target 104, track the anatomic target 104, or both.

The alignment controller 30 may send a signal to a display and/or an electronic device associated with the subject to indicate the determined on-target percent alignment is within a threshold on-target percent alignment (e.g., 70% alignment, 80%, alignment, 90% alignment) for effective therapy dosage. For example, the therapy module 12 may indicate that the therapy beam 106 is appropriately aligned for effective therapy dosage by displaying a green light or other indication to the user (e.g., subject and/or therapist) on a display of the system 10, the green light may indicate that no adjustment is needed. The energy application device may also display a signal indicating a directional adjustment in position and/or orientation to improve alignment with the anatomic target 104. The directional signal may include a degree of freedom (DOF) the therapy transducer 22 should be repositioned. It should be understood, that the signal may correspond to any visual, haptic, audible, or other guidance cues being displayed to the user. The signal may be a control signal to display an indication to the user (or otherwise notify the user) to move the energy application device to enable manual position and/or orientation adjustment by the user, a control signal to the automation controller 31 to automatically adjust the energy application device position and/or orientation by electronically steering and/or actuating one or more therapy transducers 24 and/or the probe module 14 of the energy application device, or a combination of both manual and automatic adjustment methods.

The directional signal may also indicate a user adjustment of the probe module 14 relative to the subject surface 100. The directional signal may include suggested external and/or internal adjustments to maintain a threshold percent alignment, and may provide multiple guidance cues to a user throughout the dosing session. For example, the directional signal may be sent to a user device and/or displayed on the display of the system and may instruct the user to reposition the probe 14 in an upwards direction and/or a downwards direction relative to the subject body surface 100. The directional signal may be sent from the alignment controller 30, and may indicate to the subject when the directional movement is completed to align the probe module 14 at a desired location determined by the alignment controller 30 to maintain a threshold percent alignment for effective therapy treatment. Once alignment of the device within a threshold of the anatomic target 104 is verified via communication with the alignment controller 30, the therapy transducer 22 may produce the focused therapy beam 106 to provide therapy to the anatomic target 104 for the subject.

Additionally, in some embodiments the directional signal from the alignment controller 30 may be sent to the automation controller 31 that enables movement of the probe module 14 without user input to maximize the alignment criteria provided in the directional signal of the alignment controller 30. Further, in some embodiments the alignment controller 30 may send a directional signal to the automation controller 31 to enable subject-implemented adjustment and automated adjustments to be implemented in combination to achieve the threshold percent alignment for an effective therapy dosage. The automation controller 31, upon receipt of the signal, may automatically adjust the probe module 14 position on the user, adjust an internal position of the probe transducers 20, or both to improve alignment to the anatomic target 104. The automation controller 31 may perform internal and/or external adjustments of the probe module 20 until the alignment meets a target and/or threshold alignment.

For example, the alignment controller 30 may determine the alignment is below a threshold target alignment and may send a signal to the automation controller 31 to adjust the probe module 14 position and/or orientation according to anatomic target 104 location within the image data. The alignment controller 30 may dynamically send adjustments in position and/or orientation to the automation controller 31 to correspond to patient respiration cycle, patient movement, and the like. This may ensure automatic on-target alignment of the probe module 14 so that therapy can be effectively applied.

In some embodiments, the energy application device may include a visual cue and/or signal to be output via the alignment controller 30 in combination with automatic alignment via the alignment controller 30 sending one or more signals to the automation controller 31 to adjust probe module 14 position and/or orientation. For example, the alignment controller 30 may display a red light, play an audible tone, and/or generate a haptic signal that lets the user know the energy application device does or does not meet a threshold alignment. The alignment controller 30 may also send a signal to the automation controller 31 to automatically adjust the position and/or orientation of the probe module 14 based on the alignment threshold and received images of the anatomic target 104. Any combination of manual and/or automatic alignment of the probe module 14 may be implemented to efficiently apply a therapy dose throughout a therapy session to a patient.

Additionally, the alignment controller 30 may receive sensor data in combination with image data to determine an observed trajectory and directional movement that would result in a threshold percent alignment for effective therapy dosage. The sensor data may correspond to or be generated using an inertial measurement unit (IMU) position sensor which may include one or more of accelerometers, gyroscopes, and magnetometers, optical tracker sensors, or any other suitable sensor. The alignment controller 30 may receive sensor data including data related to subject body surface motion relative to time (e.g., respiration cycle) and position of the probe 14 over time (e.g., handheld cases). It should be understood that any suitable position and/or image data may be used by the alignment controller 30 to determine suitable probe 14 position.

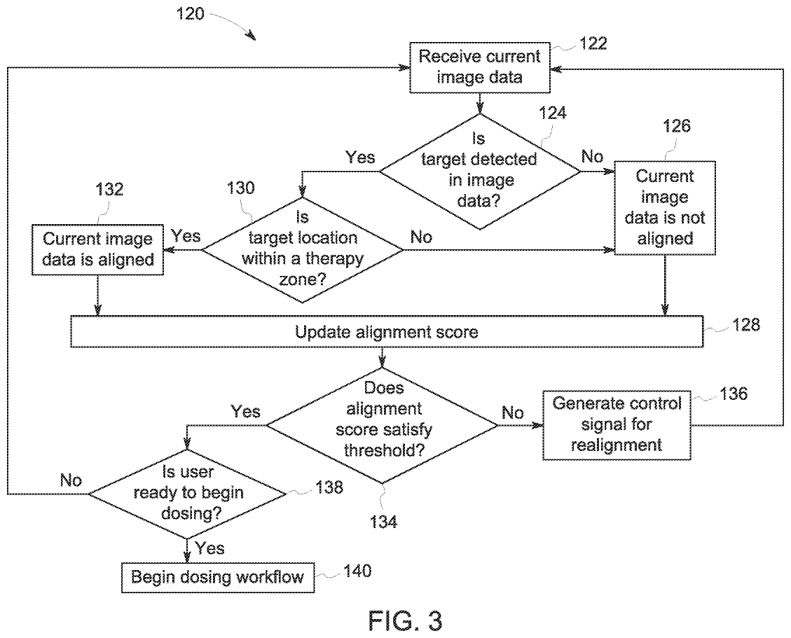

With the preceding in mind, FIG. 3 is a flow diagram of a method 120 of alignment control of the energy application device, in accordance with an embodiment of the present disclosure. The alignment controller 30 of the energy application device may receive image frame data generated via the imaging transducer 24 (e.g., ultrasound transducers) and may determine if an alignment score of the energy application device exceeds a threshold alignment score for effective therapy dose application based on the received image data.

The alignment controller 30, at block 122, receives image frame data generated by the one or more imaging transducers 24 of the energy application device. The image frame data may include image frames of internal tissue of a subject relative to the position and orientation of the energy application device to the subject body surface 100. The image frame data may include time-series data of internal tissue of a subject relative to the position of the energy application device over time. The time-series image frame data may be collected over one or more respiration cycles of a subject, or any other suitable time amount relating to subject body surface 100 movement.

The alignment controller 30, at block 124, determines if an anatomic target 104 is detected within the image data. The alignment controller 30 may determine if the anatomic target is detected in one or more frames of the image data. As discussed above, the alignment controller 30 may localize the anatomic target 104 within the received ultrasound image data by comparing the image data to previously acquired image data of the anatomic target 104 and/or previously acquired image data of the anatomical region surrounding the anatomic target 104. The alignment controller 30 may utilize the image data to extract the location of the anatomic target 104 within the ultrasound image data, and may localize the image data relative to the anatomic target 104. If the alignment controller 30 determines the anatomic target 104 is not detected within the image data the alignment controller 30, at block 126, determines that the energy application device is not aligned with the anatomic target 104. Based on the anatomic target 104 not being aligned, the alignment controller 30, at block 128, updates an alignment score.

If the anatomic target 104 is detected within the image data, the alignment controller 30, at decision block 130, determines if the anatomic target 104 is within the therapy zone 102 (e.g., region within the ultrasound image data the therapy transducer is able to deliver therapy to). In some embodiments, the alignment controller 30 may determine if the anatomic target 104 is within the therapy zone 104, and additionally, whether the anatomic target 104 is within a clinician defined contour region 110. As discussed above, in some embodiments the contour region 110 (e.g., clinician defined subject specific region that is associated with the anatomic target location) may be implemented in the decision process for therapy, and used to verify reliable alignment and prevent false positives based on inaccurate anatomic target 104 detection by the alignment controller 30. If the anatomic target 104 is detected as within the therapy zone 102 the alignment controller 30, at block 132, determines that the current image data is aligned and the alignment controller 30, at block 128, updates an alignment score associated with the energy application device position and orientation. If the alignment controller 30 determines at decision block 130, that the detected anatomic target 104 is not within the contour region 110 and the therapy zone 102, the method 120 returns to block 126, and the alignment controller 30 determines that the current image data is not aligned.

The alignment controller 30, at block 128, updates the alignment score for the energy application device based on if the current image is aligned or not aligned. As discussed above, the alignment controller 30 may determine an alignment score (e.g., percent alignment score, average percent alignment score, yes and/or no indication of alignment) indicating a level of alignment and/or alignment of the therapy zone 102 with the localized anatomic target 104. The alignment score may be calculated based on determining the therapy zone of the probe module 14 (e.g., region within the ultrasound image data the therapy transducer is able to deliver therapy to) and if the to the anatomic target 104 is within suitable range of the therapy zone 102. The alignment score may also be calculated based on if the anatomic target 104 is within a contour region 110 (e.g., clinician defined subject specific region that is associated with the anatomic target location) to verify reliable alignment and prevent false positives based on inaccurate anatomic target 104 detection by the alignment controller 30. It should be understood, that any suitable criteria may be used to calculate an alignment score using the alignment controller 30 based on the received image data and past subject data and/or clinician defined contour regions 110 within the subject image data. In some embodiments, the anatomic target 104 may be localized in one or more images over time to determine an anatomic target 104 trajectory. The observed trajectory may be compared to the energy application device beam steering capabilities to compute a predicted cumulative alignment score over the trajectory that may be used. The cumulative alignment score may correspond to one or more respiration cycles of the subject, and may be time weighted. Additionally, a predicted trajectory of the anatomic target 104 may be computed in some embodiments.

The alignment controller 30, at decision block 134 determines if the updated alignment score satisfies the predetermined threshold range. As discussed above, the predetermined threshold range may correspond to a set threshold for effective therapy dosage based on subject data. For example, an alignment score corresponding to a predetermined threshold range of 90%-100% alignment may correspond to therapy dosage application for five minutes while the alignment score is within the predetermined threshold range of 90%-100% alignment, an alignment score corresponding to a predetermined threshold range of 50%-80% alignment may correspond to therapy dosage application of 10 minutes while the alignment score is within the predetermined threshold range of 50%-80% alignment. The therapy dosage time may be dynamically adjusted based on the alignment score threshold range, so that therapy dosage application time corresponds to, or is otherwise based on, the alignment score over time. It should be understood that any suitable therapy dosage time and/or dosing level may be applied to the subject based on the predetermined threshold range of the alignment score. Additionally, the predetermined threshold range may be any suitable range based on a fully or partially predicted dose session time, a predicted therapy efficacy based on the alignment score, or both. If the alignment controller 30 determines that the updated alignment score is not within the predetermined threshold range, the alignment controller 30, at block 136, generates a control signal for realignment. The control signal for realignment may be sent based on the alignment score being outside the predetermined threshold range for greater than a certain amount of seconds (e.g., 5 seconds, 10 seconds) or a certain number of image frames (e.g., 1, 5, 10). The method 120 may then continue determining alignment scores, and when the alignment score is within the predetermined threshold range, the energy application device may begin or resume tracking and/or dosing.

The control signal may be sent to an additional controller and/or module within the master controller 80 that may determine adjustments needed to improve the alignment score. The automation controller 31 controller may then send a communication to a user device or interface that displays instructions on how to adjust the energy application device to increase the alignment score. It should be understood that any suitable control signal may be displayed and/or used to indicate adjustments to the energy application device. Additionally, the control signal may enable the energy application device to automatically steer and/or reposition to a more suitable position for effective therapy dosing. The control signal may also indicate the adjustment in probe module 14 position and orientation to align with the anatomy predicted trajectory of the target path 112. The control signal may include an adjustment to position and orientation of the probe module 14 that enables the alignment score to meet, exceed, and/or maximize the predetermined threshold range determined for the anatomic target 104. In some embodiments the trajectory of the target path 112 may be stored as subject-specific trajectory in a memory of the system 10.

If the alignment controller 30 determines that the alignment score does satisfy the predetermined threshold range, the alignment controller 30, at decision block 138, determines if a user of the energy application is ready to begin dosing. The alignment controller 30, may receive a signal that a user has input that the user is ready to begin dosing. If the alignment controller 30, receives a signal that the user is ready to begin dosing, the alignment controller, at block 140 may begin the dosing workflow. Additionally, the alignment controller 30 may send a signal to the energy application device to maintain therapy dosage if the therapy dosage session has initiated, and to correspondingly maintain position or orientation of the energy application device.

If the alignment controller 30 does not receive a notification that the user is ready to begin dosing, the method 120 returns to block 122 and continues to receive ultrasound image data of subject tissue. It should be understood that the alignment score and predetermined threshold alignment may be calculated using any suitable method for alignment calculation. It should be understood, that the method 120 may be implemented using any of the above discussed alignment score determinations.

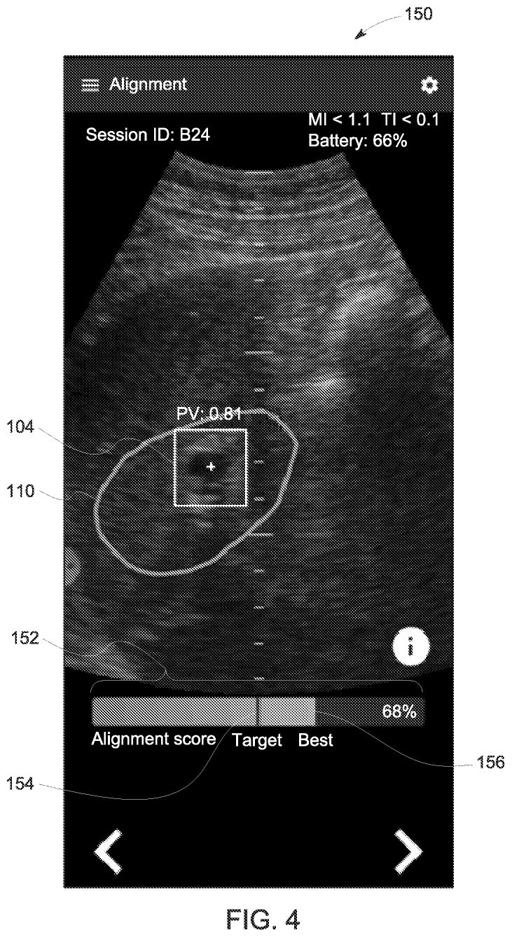

The method 120 for alignment may be used in combination with an application that may help the user guide alignment via a user interface of the application. With that in mind, FIG. 4 is an illustration of a user interface 150 for alignment control of the probe of the neuromodulation delivery system of FIG. 1, in accordance with embodiments of the present disclosure. The user may be guided by an application that includes a user interface 150 during alignment control of the energy application device. The user interface 150 may display the ultrasound images generated by the imaging transducer 22 of the probe module 14. The user interface 150 may also display an alignment score visualization 152 that includes a current alignment score for the probe module 14, and also includes a target alignment score metric 154 and a best alignment score metric 156.

At the beginning of a dosing session a user may open an application on a user device associated with the energy application device and the application may display a user interface 150 that displays updated ultrasound image data generated by the imaging transducer 22 of the energy application device. The user interface 150 may also display a box outline around the anatomic target 104 if it is detected within the image data by the alignment controller 30, and may also display an outline depicting the contour region 110 predefined by a clinician that may be additionally used to determine alignment with the determined location of the anatomic target 104 within the image data. The user interface 150 may update over time based on real-time imaging data generated by the imaging transducer 22 and analyzed by the alignment controller 30. The user interface 150 may also display an alignment score visualization 152 that may include a progress bar that corresponds to a calculated percent alignment score by the alignment controller 30. The alignment score visualization 152 may also include the target alignment score 154 for satisfactory dosage. The target alignment score 154 may be predefined by the application based on subject data and population data for treatment of subject conditions. The target alignment score 154 may also be one in the same as the predetermined threshold for effective therapy dosage. Additionally, the alignment score visualization 152 may include the best alignment score 156 marker that is set at the best alignment score achieved over a dosing session.

Further, the energy application device may guide the user based on the images displayed on the user interface 150, and whether the anatomic target 104 is within the contour region 110 overlaid on the current displayed image. The user interface 150 may also provide updates based on control signals generated by the alignment controller 30 to reposition and/or adjust the orientation and/or position of the energy application device based on the determined alignment score.

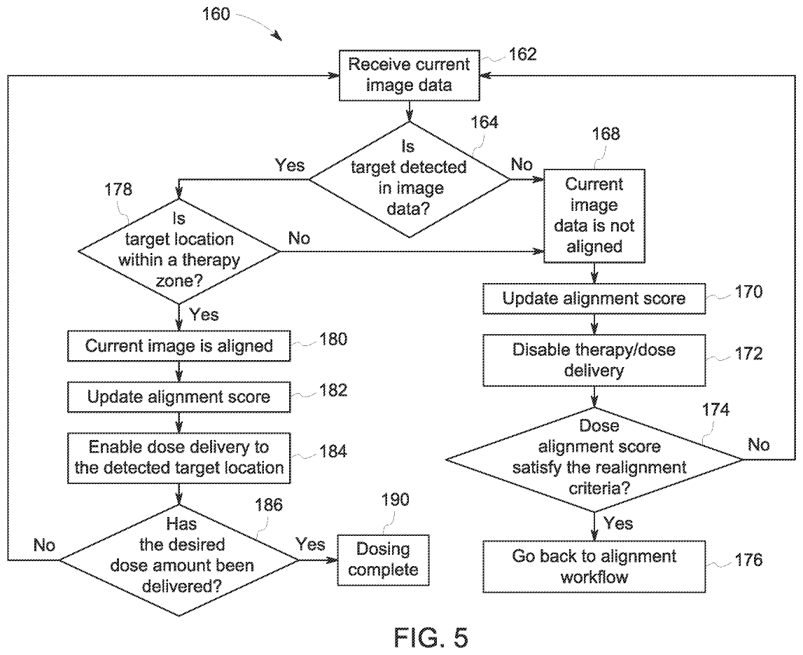

With the preceding in mind, FIG. 5 is a flow diagram of a method 160 of delivering a therapy dose via the probe module 14 of the neuromodulation delivery system 10 (e.g., system), in accordance with embodiments of the present disclosure. The alignment controller 30 of the energy application device may receive image frame data generated via the imaging transducer 24 (e.g., ultrasound transducers) and may determine if a determined alignment score of the energy application device exceeds a threshold alignment for effective therapy dose application based on the received image data, and then may enable dosing to begin and monitor alignment throughout a dosing session.

The alignment controller 30, at block 162, receives image frame data generated by the one or more imaging transducers 24 of the energy application device. The image frame data may include image frames of internal tissue of a subject relative to the position and orientation of the energy application device to the subject body surface 100. The image frame data may include time-series data of internal tissue of a subject relative to the position of the energy application device over time. The time-series image frame data may be collected over one or more respiration cycles of a subject, or any other suitable time amount relating to subject body surface 100 movement.

The alignment controller 30, at decision block 164, determines if an anatomic target 104 is detected within the image data. The alignment controller 30 may determine if the anatomic target 104 is detected in one or more frames of the image data. As discussed above, the alignment controller 30 may localize the anatomic target 104 within the received ultrasound image data by comparing the image data to previously acquired image data of the anatomic target 104 and/or previously acquired image data of the anatomical region surrounding the anatomic target 104. The localization of the anatomic target 104 may be determined based on subject specific image data and/or based on population-based image data corresponding to the anatomical region surrounding the anatomic target 104. The alignment controller 30 may utilize the image data to extract the location of the anatomic target 104 within the ultrasound image data, and may localize the image data relative to the anatomic target 104. If the alignment controller 30 determines the anatomic target 104 is not detected within the image data, the alignment controller 30, at block 168, determines that the energy application device is not aligned with the anatomic target 104. Based on the anatomic target 104 not being aligned, the alignment controller 30, at block 170 updates an alignment score.

The alignment controller 30, at block 172, sends a control signal to disable therapy in response to the therapy dose alignment being misaligned. The alignment controller 30, at decision block 174, determines if the alignment score satisfies one or more realignment criteria. The realignment criteria may include a numerical threshold for the alignment score, a threshold percent on-target alignment of the alignment score over time, or any other suitable numerical and/or time dependent alignment criteria. For example, if the alignment score is 20% less than the predetermined alignment score threshold, the realignment criteria may be met, and the alignment controller 30 may send a signal to trigger realignment. In another example, if the alignment score is determined to be less than the predetermined threshold range for greater than 5 consecutive seconds, the alignment controller 30 may send a signal to trigger realignment.

For example, the alignment score during a session may still be above a predetermined threshold range, and the dosing session may continue. If the alignment score does not satisfy the realignment criteria, the method 160 returns to block 162 and receives current image data. If the alignment score satisfies the realignment criteria, the alignment controller 30 may, at block 176, return to the alignment workflow.

Returning to block 164, if the anatomic target 104 is detected within the image data, the alignment controller 30, at decision block 178, determines if the anatomic target 104 is the therapy zone 102 (e.g., region within the ultrasound image data the therapy transducer is able to deliver therapy to). As discussed above, the contour region 110 (e.g., clinician defined subject specific region that is associated with the anatomic target location) may also be used in combination with the therapy zone 102 to verify reliable alignment and prevent false positives based on inaccurate anatomic target 104 detection by the alignment controller 30. If the anatomic target 104 is detected as within the contour region 110 and/or the therapy zone 102 the alignment controller 30, at block 180, determines that the current image data is aligned and the alignment controller 30, at block 182, updates an alignment score associated with the energy application device position and orientation. If the alignment controller 30 determines at decision block 178, that the detected anatomic target 104 is not within the contour region 110 and/or the therapy zone 102 the method 120 returns to block 168, and the alignment controller 30 determines that the current image data is not aligned.

The alignment controller 30, at block 182, updates the alignment score for the energy application device based on the image alignment. As discussed above, the alignment controller 30 may determine an alignment score (e.g., percent alignment score, average percent alignment score, yes and/or no indication of alignment) indicating a level of alignment and/or alignment of the therapy zone 102 with the localized anatomic target 104. Additionally, the alignment score may correspond to one or more alignment criteria, that may include one or more alignment scores associated with a threshold alignment score for a yes and/or no indication of alignment. The alignment criteria may also correspond to a combination of alignment scores and one or more checklist items associated with probe alignment. The checklist and alignment scores may be used in combination as alignment criteria to determine a yes and/or no indication of alignment.

The alignment score may be calculated based on determining the therapy zone of the probe module 14 (e.g., region within the ultrasound image data the therapy transducer is able to deliver therapy to) and if the anatomic target 104 is within suitable range of the therapy zone 102. The alignment score may also be calculated based on if the anatomic target 104 is within a contour region 110 (e.g., clinician defined subject specific region that is associated with the anatomic target location) and the therapy zone 102 to verify reliable alignment and prevent false positives based on inaccurate anatomic target 104 detection by the alignment controller 30. It should be understood, that any suitable criteria may be used to calculate an alignment score using the alignment controller 30 based on the received image data and past subject data and/or clinician defined contour regions 110 within the subject image data.

The alignment controller 30, at block 184, enables dose delivery to the anatomic target 104. The dose may be delivered based on the alignment score and a predetermined threshold range the alignment score corresponds to. For example, the predetermined threshold range may correspond to a set threshold for effective therapy dosage based on subject data. For example, an alignment score corresponding to a predetermined threshold range of 90%-100% alignment may correspond to therapy dosage application for five minutes, an alignment score corresponding to a predetermined threshold range of 50%-80% alignment may correspond to therapy dosage application of 10 minutes. It should be understood that any suitable therapy dosage time and/or dosing level may be applied to the subject based on the predetermined threshold range of the alignment score. Additionally, the predetermined threshold range may be any suitable range based on a fully or partially predicted dose session time, a predicted therapy efficacy based on the alignment score, or both. In some embodiments, a dosing controller may receive information from the alignment controller 30 including determined location of the anatomy region 104, alignment score, predetermined threshold range, and the control signal. The dosing controller may send a signal for the therapy transducer 24 to begin dosing when the control signal indicates the anatomic target 104 is aligned and/or the alignment score is within the predetermined threshold range. The loop time to localize the anatomic target 104 within the image frames, determine the control signal and activate the therapy beam to direct therapy towards the localized anatomic target 104 may be less than 500 milliseconds or any other suitable loop time. In some embodiments, the dosing controller may accumulate a measure or estimate of the total dose applied (e.g., number of pulses, time of dosing, accumulated energy of dosing) to the anatomic target 104. The dosing controller may send a signal when the amount of accumulated dose meets a threshold amount of dose needed for therapy efficacy. In certain embodiments the dosing controller may be housed within the master controller 80 of the energy application device.

The alignment controller 30, at decision block 186, determines if the desired dose amount has been completed. If the alignment controller 30 determines that the desired dosage amount has not yet been delivered, the method 160 returns to block 162. If the alignment controller 30, determines that the desired dose amount has been delivered the alignment controller 30, at block 190, determines that the dosing session is complete, and may send a signal to the user device to indicate that dosing is complete.

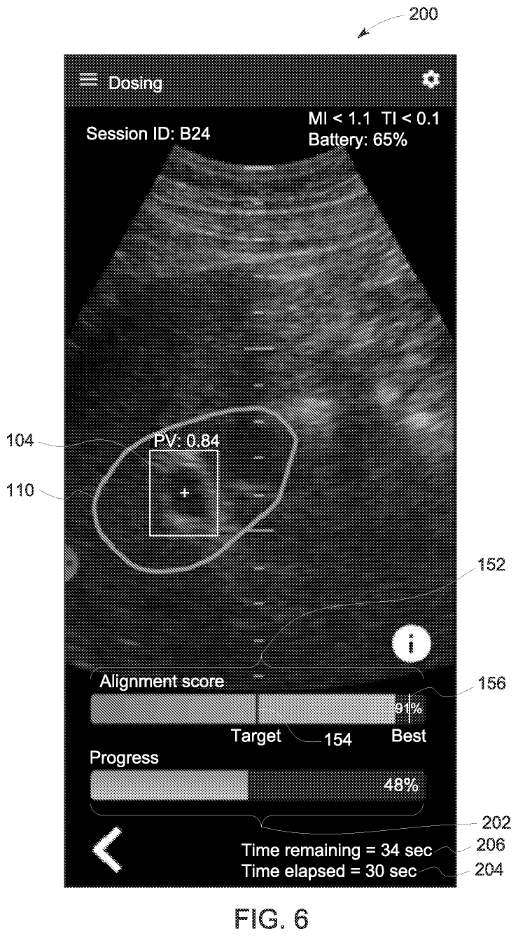

The user may be guided throughout the dosing session using an application of the user device and/or display of the energy application device. For example, FIG. 6 is an illustration of a dosing user interface 200 for therapy dosing via the probe of the neuromodulation delivery system 10, in accordance with embodiments of the present disclosure. The user may be guided by an application that includes the dosing user interface 200 during dosing using the energy application device. The dosing user interface 200 may display the ultrasound images generated by the imaging transducer 22 of the probe module 14. The dosing user interface 200 may also display the alignment score visualization 152 that includes a current alignment score for the probe module 14, and also includes the target alignment score metric 154 and the best alignment score metric 156. Additionally, the dosing user interface 200 may display a progress visualization 202 corresponding to progress of the dosing throughout the dosing session, and a display of an estimated remaining dosing time 206 and the elapsed time 204.

At the beginning of a dosing session a user may open an application on a user device associated with the energy application device and the application may display a dosing user interface 200 that displays updated ultrasound image data generated by the imaging transducer 22 of the energy application device. The dosing user interface 200 may also display a box around the anatomic target 104 if it is detected within the image data by the alignment controller 30, and may also display an outline indicating the contour region 110 predefined by a clinician that may be additionally used to determine alignment with the determined location of the anatomic target 104 within the image data. The dosing user interface 200 may update over time based on real-time imaging data generated by the imaging transducer 22 and analyzed by the alignment controller 30. The dosing user interface 200 may also display an alignment score visualization 152 that may include a progress bar that corresponds to a calculated percent alignment score by the alignment controller 30. The alignment score visualization 152 may also include a target alignment score 154 for satisfactory dosage. The target alignment score 154 may be predefined by the application based on subject data and population data for treatment of subject conditions. Additionally, the alignment score visualization 152 may include a best alignment score 156 marker that is set at the best alignment score achieved over a dosing session. The progress visualization 202 may correspond to the dosing progress throughout the session based on the alignment score and predefined dosage times based on predetermined threshold alignment corresponding to alignment scores throughout the dosing session. The dosing progress throughout the session may correspond to a number of pulses or total dose time until a target number of pulses or dose time is accumulated.

Further, the energy application device may guide the user based on the images displayed on the user interface 150, and whether the anatomic target 104 is within the contour region 110 overlaid on the current displayed image. The user interface 150 may also provide updates based on control signals generated by the alignment controller 30 to reposition and/or adjust the orientation and/or position of the energy application device based on the determined alignment score. The dosing user interface 200 may also display the time remaining in a dosing session 206.

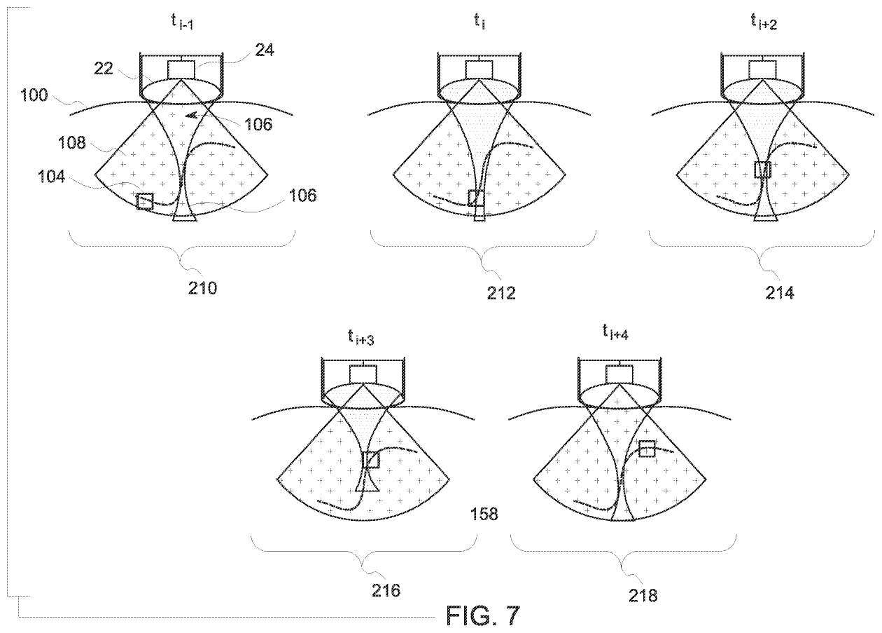

With the foregoing in mind, FIG. 7 is a schematic diagram of tracking over time using the therapy transducer 22 that has adjustable focus in one-dimension, in accordance with embodiments of the present disclosure. The energy application device may be able to adjust the therapy transducer 22 focus over time based on the anatomic target 104 position within the image frames generated by the imaging transducer 24. The alignment controller 30 may determine the therapy transducer 22 focus for the therapy beam 106 to provide effective therapy to the anatomic target 104 over the therapy session.

In some embodiments the therapy transducer 22 enables one-dimensional tracking of the anatomic target 104. For example, the therapy transducer 22 may point in a fixed direction and may have the ability to adjust focus in one-dimension. The focus adjustment may correspond to depth and may be adjusted via electronic focusing control and may adjust the therapy beam 106 through the x-axis, y-axis, and angle adjustment axis. The therapy transducer 22 focus may also be adjusted via mechanical control.

The alignment controller 30 may detect when the anatomic target 104 is within the focusing range of the therapy transducer 22 and when the therapy beam 106 is aligned with the nominal direction of the anatomic target 104. The system 10 may configure the depth of the therapy transducer 22 and may apply therapy when the anatomic target 104 is within steering range of the therapy transducer 22. The system 10 may not apply therapy or stop applying therapy when alignment with the anatomic target 104 is lost, and when the alignment is within range of the therapy transducer 22 at a later point, the system 10 may resume applying therapy.

For example, at an initial time 210, based on the imaging transducer data 24, the alignment controller 30 may determine the anatomic target 104 may not be within the focusing range of the therapy transducer 22 and the system 10 may not apply therapy. The alignment controller 30 may detect, at a second time 212 that the anatomic target 104 is within the focusing range of the therapy transducer 22, but needs to be focused to an increased depth. The alignment controller 30 may send a signal to the therapy transducer 22 to increase therapy beam 106 depth, and may send a signal to apply therapy to the system 10 so that therapy may be applied to the anatomic target 104 via the therapy transducer 22. At a third time 214, the alignment controller 30 may determine that the anatomic target 104 is within the focusing range of therapy transducer 22, but may need to decrease depth of therapy beam 106. The alignment controller 30 may then send a signal to the therapy transducer 22 to reduce depth of the therapy beam 106, and the system 10 may continue to apply therapy. At a fourth time 216, the alignment controller 30 may determine that the depth of the therapy beam 106 must be further reduced to correspond to the current position of the anatomic target 104 based on generated image data. The alignment controller 30 may send a signal to the therapy transducer 22 to decrease depth of the therapy beam 106, and the system 10 will continue to apply therapy. At a fifth time 218, the alignment controller 30 may determine that the anatomic target 104 is not within focusing range of the therapy transducer 22 and may send a control signal to the system 10 to stop therapy application. It should be understood that the therapy transducer 22 may adjust focus in one-dimension relative to the detected position of the anatomic target 104.

In some embodiments, during dosing the alignment controller 30 may send a signal to loop this adjustment pattern relative to a respiration cycle of the subject and/or may send updated signals throughout the therapy dosing session based on the detected respiration cycle of the subject. The probe module 14 may include one or more IMU sensors that collect IMU data over time for the subject during the dosing session. The IMU sensors may include one or more of accelerometers, gyroscopes, and magnetometers, or any combination of suitable sensor types. The IMU data may be used to track the anatomic target 104 movement within the subject throughout the subject's respiration cycle. The alignment controller 30 may receive the IMU data, and determine changes to position and orientation of the probe module 14 relative to the subject's respiration cycle to provide on target dosing throughout the subject's respiration cycle.

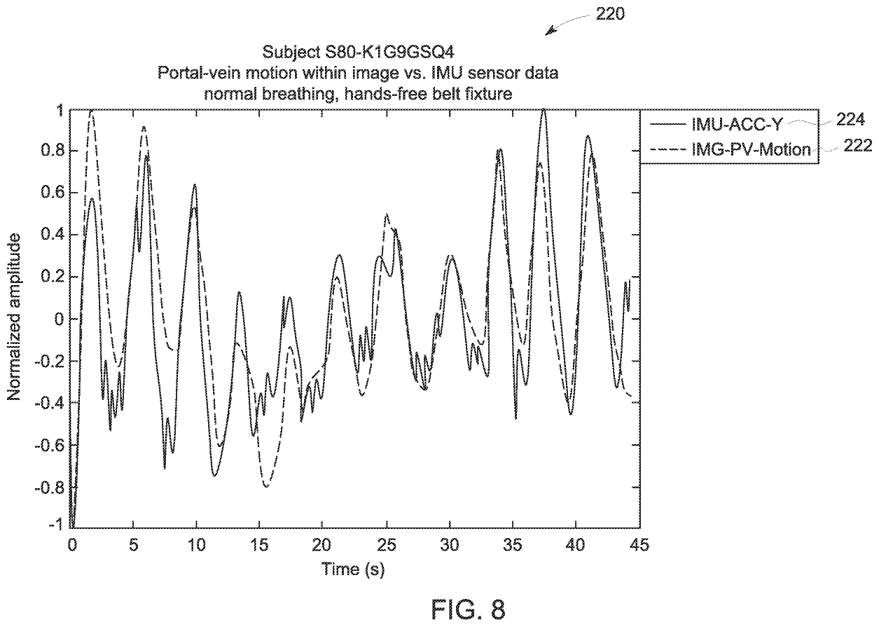

With the foregoing in mind, FIG. 8 is a graphical representation 220 of correlation of the observed motion of the portal-vein within the ultrasound image plane and motion of the energy application device based on the IMU sensor data, in accordance with embodiments of the present disclosure. The IMU data 224 may be acquired using the probe module 14 (e.g., attached to the IMU) held on the subject body surface 100. The probe module 14 may implement a synchronization sequence consisting of rocking the probe module 14 one or more times during a synchronization sequence, while the subject completes one or more respiration cycles.

The graph displays time in seconds on the x-axis, and normalized amplitude relative to the sensor data on the y-axis. The graph depicts the normalized amplitude of the portal-vein motion 224 within the ultrasound image data over time and the IMU sensor data 222 representing the probe module 14 movement over time. The graph depicts a synchronization sequence, a normal breathing cycle of five breaths, and an additional synchronization sequence. The IMU sensor data is compared to the accelerometer output for the probe module 14. The motion of the portal vein 222 is calculated as the pixel distance of the center of an annotated anatomy region 104 for each image frame versus the center of the annotated anatomy region 104 on the first frame received. The portal-vein motion data 222 and IMU sensor motion data 224 are time-adjusted, cross correlated to align, direct current (DC) bias removed, and normalized in the displayed graph.

The graph demonstrates leveraging the IMU sensor to track the internal anatomic target motion and predicted path of the anatomic target 104 during subject respiration. It should be understood that multiple embodiments may be implemented based on multiple data fusion algorithms, reduced image complexity based on sensor tracking, or any other suitable tracking method for monitoring an anatomic target 104 motion during subject respiration. In some embodiments, the IMU position sensor 40 may assist in detecting motion of the anatomic target 104 and/or detecting respiration cycles in the subject.

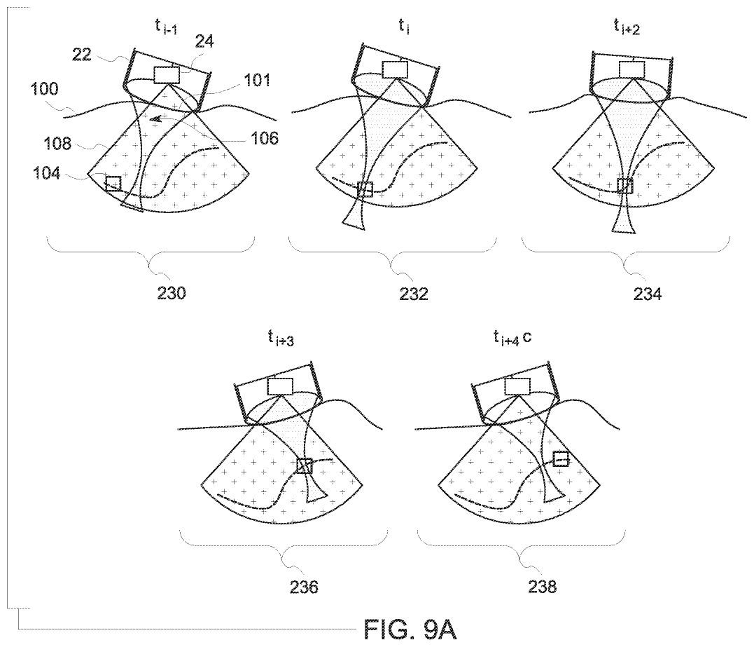

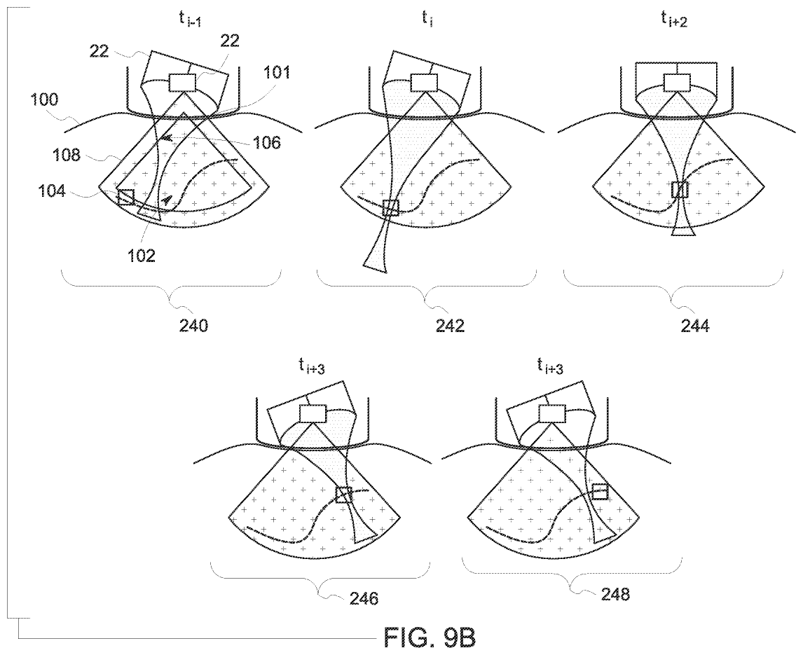

With the foregoing in mind, FIG. 9A is a schematic diagram of tracking over time using a fixed two-dimensional imaging transducer 24 with a mechanically rocked therapy transducer 22 with external motion adjustment, in accordance with embodiments of the present disclosure. In some embodiments, the therapy transducer 22 may be mechanically rocked (e.g., pivot within the azimuthal plane) via motor control and include axial electronic focusing of the therapy transducer 22. The anatomic target 104 within the image plane 108 may be tracked by the alignment controller 30. Additionally, the alignment controller 30 may determine when the anatomic target 104 is within the therapy zone 102 (e.g., the area around which the therapy transducer may pivot to apply therapy) of the therapy transducer 22. The alignment controller 30 may then send a signal to the therapy probe module 14 to mechanically rock towards the anatomic target 104 and/or may send a signal to adjust the therapy beam 106 depth electronically to focus the therapy beam 106 at the anatomic target 104 depth. The therapy transducer 22 may be external to the lens 101 of the energy application device, and the therapy transducer 22 may pivot using external motion relative to the lens 101. It should be understood that the therapy transducer 22 may adjust focus in one-dimension relative to the detected position of the anatomic target 104. The alignment controller 30 may send a signal to loop this adjustment pattern relative to a respiration cycle of the subject and/or may send updated signals throughout the therapy dosing session.

For example, at an initial time 230, based on the imaging transducer data 24, the alignment controller 30 may determine the anatomic target 104 may not be within the therapy zone 102 of the therapy transducer 22 and the system 10 may not apply therapy. The alignment controller 30 may detect, at a second time 232, that the anatomic target 104 is within the therapy zone 102 of the therapy transducer 22, but adjustment is needed to focus the therapy beam 106 by pivoting the probe module 14 to the left in the azimuthal plane and increasing the depth of the therapy beam 106 using axial electronic focusing to apply therapy to the anatomic target 104. The alignment controller 30 may send a signal to the probe module 14 (e.g., using actuators within the probe module 14) to pivot to the left and increase focusing depth, and may send a signal to the system 10 to apply therapy so that therapy may be applied to the anatomic target 104. At a third time 234, the alignment controller 30 may determine that the anatomic target 104 is within the therapy zone 102 of the therapy transducer 22, but adjustment is needed to focus the therapy beam 106 by sending a signal to the probe module 14 to pivot right in the azimuthal plane to apply therapy to the anatomic target 104. The alignment controller 30 may then send a signal to the probe module 14 to pivot to the right, and the system 10 will continue therapy. At a fourth time 236, the alignment controller 30 may determine that the depth of the therapy beam 106 focus may need to be further reduced to correspond to the anatomic target 104 based on generated image data and the probe module 14 may need to pivot to the right. The alignment controller 30 may send a signal to the therapy transducer 22 to decrease depth of the therapy beam 106 and for the probe module 14 to pivot to the right, and for the system 10 to maintain therapy application. At a fifth time 238, the alignment controller 30 may determine that the anatomic target 104 is not within the therapy zone 102 of the therapy transducer 22 and may send a control signal to the system 10 to stop applying therapy. It should be understood that the therapy transducer 22 may adjust focus relative to the detected position of the anatomic target 104. The alignment controller 30 may send a signal to loop this adjustment pattern relative to a respiration cycle of the subject and/or may send updated signals throughout the therapy dosing session. That is, the alignment controller 30 may send a signal to continually apply therapy dosage throughout the time during the therapy session during which the alignment score meets a threshold alignment score. It should be understood that the alignment controller 30 may provide any suitable signal based on the determined alignment of the energy application device (e.g., “aligned”, “not aligned”, “trending toward alignment”, “trending away from alignment”, “in position, but not oriented correctly”, and so forth). That is, the signal may be a signal to maintain position and/or orientation of the energy application device, maintain therapy dosage, stop therapy dosage, and/or make an adjustment to position and/or orientation of the energy application device.

With the foregoing in mind, FIG. 9B is a schematic diagram of tracking control over time using a fixed two-dimensional imaging transducer 24 with a mechanically rocked therapy transducer 22 with internal motion adjustment, in accordance with embodiments of the present disclosure. In some embodiments, the therapy transducer 22 may be mechanically rocked (e.g., pivot within the azimuthal plane) via motor control and include axial electronic focusing of the therapy transducer 22. The anatomic target 104 within the image plane 108 may be tracked by the alignment controller 30. Additionally, the alignment controller 30 may determine when the anatomic target 104 is within the therapy zone 102 of the therapy transducer 22. The alignment controller 30 may then send a signal to the probe module 14 (e.g., actuators within the prove module 14) to mechanically rock towards the anatomic target 104 and/or may send a signal to adjust the therapy beam 106 depth electronically to focus the therapy beam 106 at the anatomic target 104 depth. The therapy transducer 22 may be internal to the lens 101 of the energy application device, and the therapy transducer 22 may pivot using internal motion relative to the lens 101. It should be understood that the therapy transducer 22 may adjust focus in one-dimension relative to the detected position of the anatomic target 104. The alignment controller 30 may send a signal to loop this adjustment pattern relative to a respiration cycle of the subject and/or may send updated signals throughout the therapy dosing session

As discussed above, the probe module 14 may be automatically and/or mechanically steered or focused over time based on the anatomic target 104 location relative to the therapy transducer. In a similar manner the therapy transducer 24 may be mechanically rocked using internal motion and/or focused over time 240, 242, 244, 246, 248 to achieve alignment with the anatomic target 104.

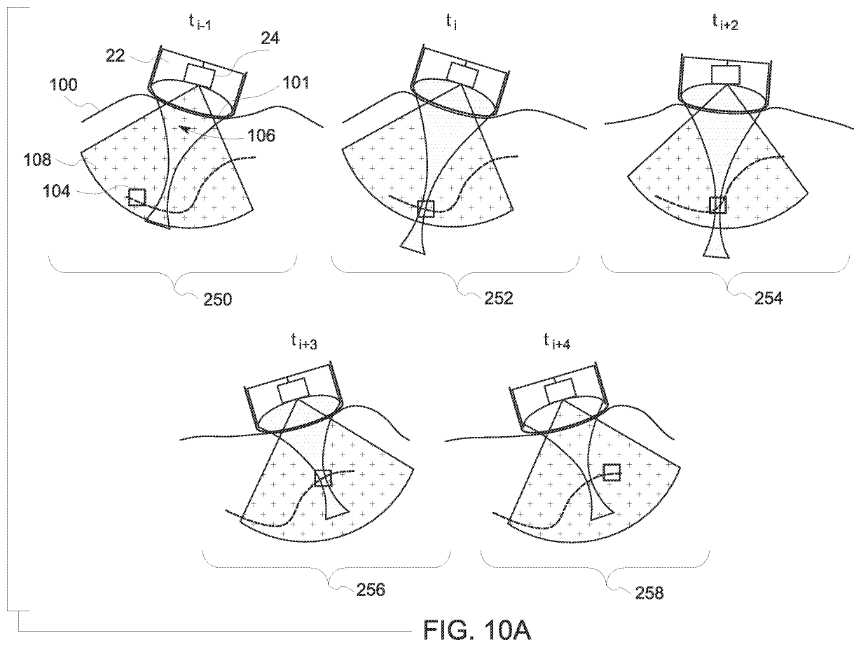

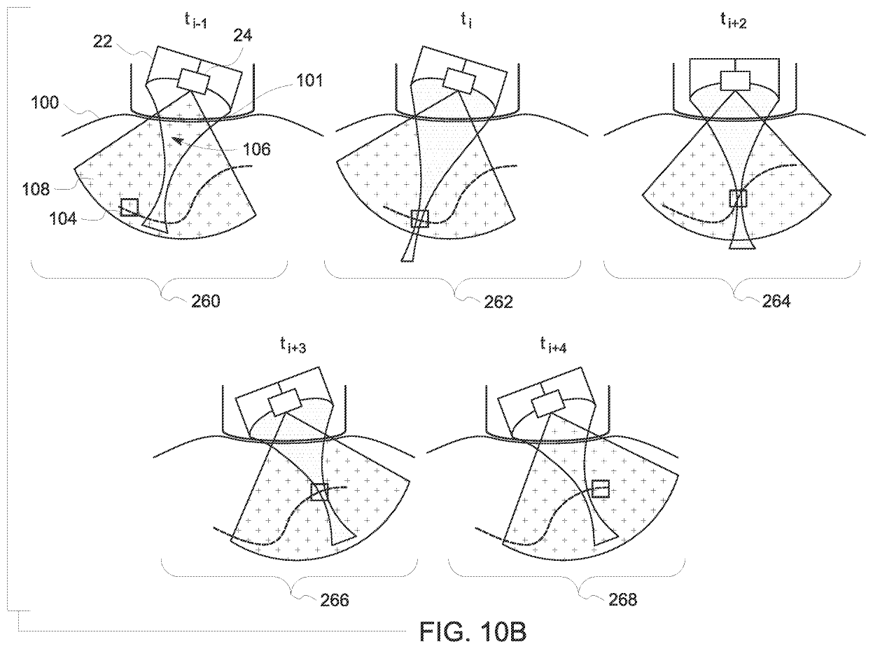

With the foregoing in mind, FIG. 10A is a schematic diagram of tracking over time using a two-dimensional imaging transducer 24 coupled to the therapy transducer 22 with external motion adjustment, in accordance with an embodiment of the present disclosure. The energy application device may include the two-dimensional imaging transducer 24 and the therapy transducer 22 that are fixed relative to each other, and mechanically rocked via motor control and electronically focused relative to tracking of a subject anatomic target 104. The system 10 may rock the transducer assembly 22, 24 that includes the imaging transducer 24 and the therapy transducer 22 to maintain the anatomic target 104 alignment throughout dosing. The method of motion of the transducer assembly 22, 24 may be external to the lens 101 of the energy application device. It should be understood that the transducer assembly 22, 24 may adjust focus relative to the detected position of the anatomic target 104. The alignment controller 30 may send a signal to loop this adjustment pattern relative to a respiration cycle of the subject and/or may send updated signals throughout the therapy dosing session.

For example, at an initial time 250, based on the imaging transducer data 24, the alignment controller 30 may determine the anatomic target 104 may not be within the therapy zone 102 of the therapy transducer 22 and the system 10 may stop applying therapy. The alignment controller 30 may detect at a second time 252 that the anatomic target 104 is within the therapy range 102 of the therapy transducer 22, but to maintain focus, the probe module 14 is sent a signal to pivot to the left in the azimuthal plane and increase the depth of the therapy beam 106 with axial electronic focusing to apply therapy to the anatomic target 104. The alignment controller 30 may send a signal to the probe module 14 to pivot to the left and increase focusing depth, and may send a signal to the system 10 to apply therapy so that therapy may be applied to the anatomic target 104. At a third time 254, the alignment controller 30 may determine that the anatomic target 104 is within the therapy zone 102 of the therapy transducer 22, and to focus the transducer assembly 22, 24, the assembly is pivoted to the right in the azimuthal plane to apply therapy to the anatomic target 104. The alignment controller 30 may then send a signal to the probe module 14 to pivot to the right, and the system 10 may continue to apply therapy. At a fourth time 256, the alignment controller 30 may determine that the depth of the therapy beam 106 must be further reduced to correspond to the location of the anatomic target 104 based on generated image data, and the transducer assembly 22, 24 may pivot to the right. The alignment controller 30 may send a signal to the probe module 14 to decrease depth of the therapy beam 106 and pivot to the right, and the system 10 will continue to apply therapy. At a fifth time 258, the alignment controller 30 may determine that the anatomic target 104 is not within the therapy zone 102 of the therapy transducer 22 and may send a control signal to the system 10 to stop applying therapy. It should be understood that the transducer assembly 22, 24 may adjust focus relative to the detected position of the anatomic target 104. The alignment controller 30 may send a signal to loop this adjustment pattern relative to a respiration cycle of the subject and/or may send updated signals throughout the therapy dosing session.

FIG. 10B is a schematic diagram of tracking over time using a two-dimensional imaging transducer 24 coupled to the therapy transducer 22 adjustment with internal motion adjustment, in accordance with an embodiment of the present disclosure. The energy application device may include a two-dimensional imaging transducer 24 and the therapy transducer 22 that are fixed relative to each other, and mechanically rocked via motor control and electronically focused relative to tracking of a subject anatomic target 104. The system 10 may rock the transducer assembly 22, 24 that includes the imaging transducer 24 and the therapy transducer 22 to maintain the anatomic target 104 alignment in a fixed direction. The method of motion of the transducer assembly 22, 24 may be internal to the lens 101 of the energy application device. As discussed above, the therapy transducer may be automatically and/or mechanically steered or focused over time based on the anatomic target 104 location relative to the transducer assembly 22, 24. In a similar manner the transducer assembly 22, 24 may be mechanically rocked using internal motion and/or focused over time 260, 262, 264, 266, 268 to achieve alignment with the anatomic target 104.

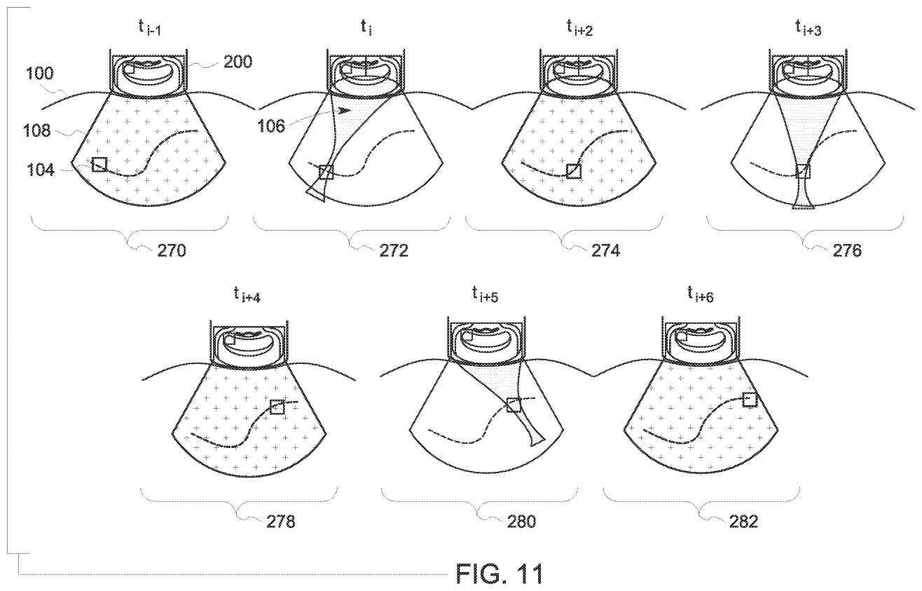

Keeping the foregoing in mind, FIG. 11 is a schematic diagram of tracking over time using a single linear transducer 200 for imaging and therapy, in accordance with an embodiment of the present disclosure. The energy application device may include an imaging transducer 22 and a therapy transducer 24 that are combined in a single component (e.g., linear transducer assembly) 200. In other embodiments, the imaging transducer 22 and therapy transducer 24 comprising the linear transducer assembly 200 are one in the same. The liner transducer assembly 200 may include an electronic steering beam in the azimuth plane for both the imaging and therapy elements of the linear imaging transducer 200. The illustrations depict interleaved imaging and therapy via the single linear transducer 200.

For example, at an initial time 270, the imaging frame 108 based on the imaging transducer 22 is shown at an initial time. The alignment controller 30 may detect at a second time 272 that the linear transducer beam is within a therapy range of the anatomic target 104 based on the imaging frame 108 at time 270, and send a signal to electronically steer the beam to the left in the azimuthal plane and increase the depth of the therapy beam 106 with axial electronic focusing to apply therapy to the anatomic target 104. In some embodiments, the personalized contour 110 region affects therapy dosage, such that therapy is not applied when the location of the anatomic target 104 is detected outside of the contour region 110. In these embodiments, when the location of the region of interest is additionally within the contour region 110, then the therapy beam 106 may be focused and steered to the location of the anatomic target 104.

Additionally, the alignment controller 30 may send a signal to the linear transducer assembly 200 to steer to the left and increase focusing depth, and a signal may be sent to the system 10 to apply therapy so that therapy may be applied to the anatomic target 104. At a third time 274, the imaging frame 108 may depict the anatomic target 104 is within the therapy zone 102. At a fourth time 276 the alignment controller 30 may send a signal to focus the linear transducer assembly 200 by electronically steering to the right in the azimuthal plane to apply therapy to the anatomic target 104. The alignment controller 30 may then send a signal to the linear transducer assembly 200 to steer to the right, and the system 10 may be sent a signal to apply therapy. At a fifth time 278, the alignment controller 30 may receive imaging frames 108 that indicate the anatomic target 104 is within the therapy zone 102. The alignment controller 30, at a sixth time 280, may send a signal to the linear transducer assembly 200 to decrease depth of the therapy beam 106 and send a signal to electronically steer to the right, and the system 10 will continue to apply therapy. At a seventh time 282, the alignment controller 30 may determine that the anatomic target 104 is not within the therapy zone based on the imaging frame data. It should be understood that the linear transducer assembly 200 may adjust focus relative to the detected position of the anatomic target 104. The alignment controller 30 may send a signal to loop this adjustment pattern relative to a respiration cycle of the subject and/or may send updated signals throughout the therapy dosing session.

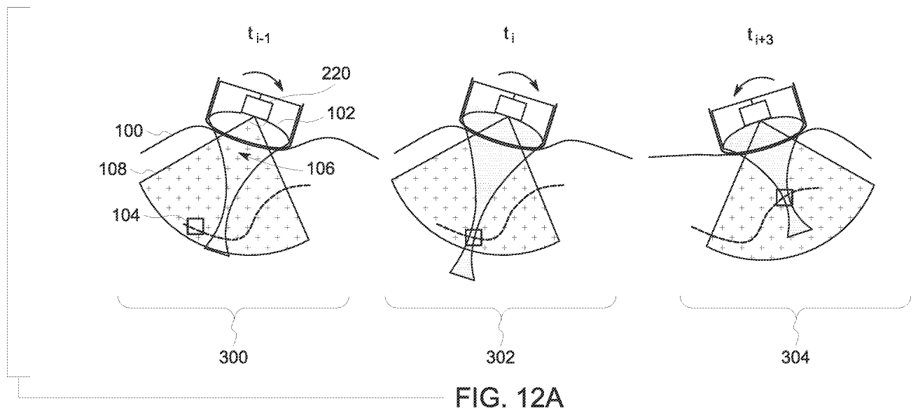

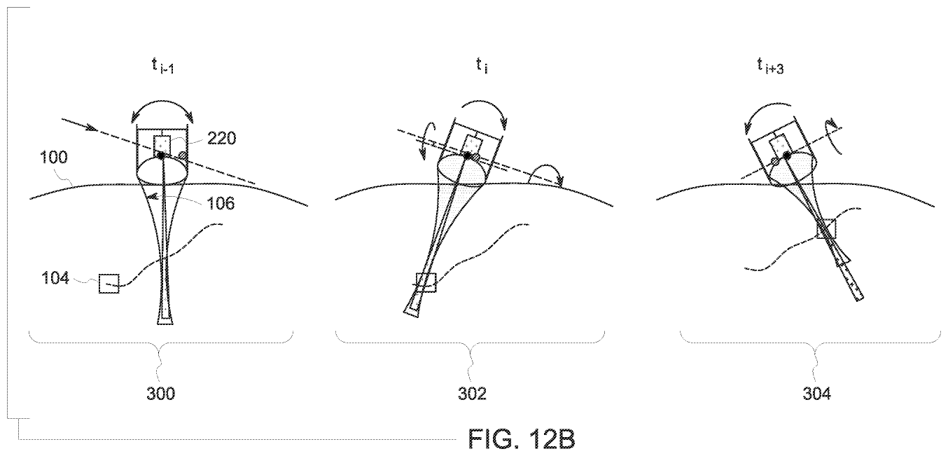

Keeping the foregoing in mind, FIG. 12A is a schematic diagram of three-dimensional tracking with movement depicted around the elevation axis, while FIG. 12B is a schematic diagram of three-dimensional tracking with movement depicted around the azimuth axis in accordance with an embodiment of the present disclosure. Together FIG. 12A and FIG. 12B illustrate aligning to and tracking a three-dimensional target path 106 over time by adjusting the energy application device in both the azimuth and elevation axis simultaneously. In the three-dimensional tracking, the entire probe module 14 may remain fixed on the subject body surface 100 with internal rotation of the therapy transducer 220 about the elevation and azimuth axes, or the probe module 14 may move relative to the subject body surface 100 with a gimbal-based (or any other mechanical-based configuration or linkage for motion) mechanism used to externally rotate the therapy transducers 220 about the elevation and azimuth axes. The elevation and azimuth axis rotations are internally or externally adjusted to align to and track the anatomic target 104 along a three-dimensional target path 106. In addition, in some embodiments, the therapy transducers 220 may have two-dimensional electronic steering and focusing capabilities around the azimuth axis.

The adjustments around the azimuth and elevation axes may include electronically controlling a therapy transducer 220 array and utilizing internal or external motors of the system 10 to reposition the therapy transducer 220 based on anatomic target 104 position relative to the energy application device over time. For example, at an initial time 300 the alignment controller 30 may detect based on imaging data that the anatomic target 104 may not be within the therapy zone 102 of the therapy transducer 220. The alignment controller 30 may detect at a second time 302 that the anatomic target 104 is within the therapy range 102 of the therapy transducer 220 and may send a control signal for the therapy transducers 220 to rotate to the left along the azimuth axis via motor control to align to the detected target location 104, and may send a control signal to the system 10 to apply therapy. At a third time 304, the alignment controller 30 may send a control signal for the therapy transducers 220 to rotate via motor control to the right along the azimuth axis and to the right along the elevation axis, and continue to power on the system 10 to provide therapy to the anatomic target 104.

Further, the gimbal assembly may include imaging in a two-dimensional focus with electronic therapy that enables adjusting the beam focus in two-dimensions with slight external tilting motion of the entire probe assembly that follows a signal corresponding to subject body 100 movement during respiration. In another embodiment, the gimbal assembly may include a swash plate concept drive system that includes a main rotor axis that enables the transducers to rock in the azimuth plane, and swash plates with control rods that enable control of the tilt and spin of the transducers 220. In another embodiment the gimbal may include a parallel mechanism with spherical and universal joints that enables movement of the therapy transducer. It should be understood that any tracking method may be applied to track subject anatomic target 104 over time.

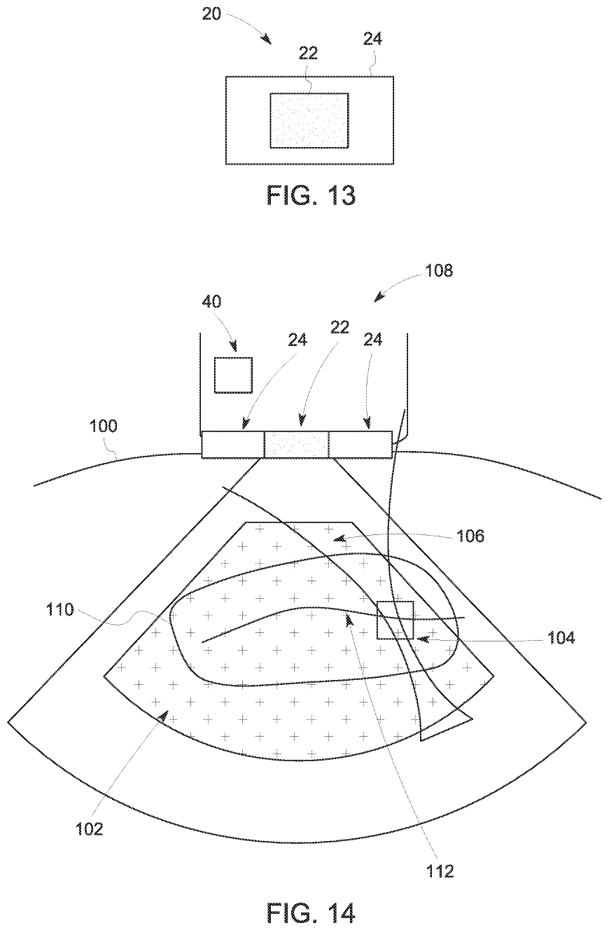

With the foregoing in mind, FIG. 13 is an illustration of a view of a transducer face relative to a subject body surface 100. The transducer 20 may include the two-dimensional imaging array 22 and the two-dimensional therapy array 24. In this embodiment, the two-dimensional imaging array 22 and the two-dimensional therapy array 24 may be capable of steering and/or focusing in three-dimensions. Specifically, when using three-dimensional focusing and steering, the image plane is an image volume, and the therapy zone 102 is a three-dimensional therapy zone. Therefore, the target path 112 may be represented in three-dimensional space and the contour region 110 may be represented as a volume. This may enable the therapy beam 106 to be steered and/or focused in three-dimensions relative to the therapy zone 102 and contour volume.

With the foregoing in mind, FIG. 14 is a schematic diagram of a fixed arrangement of imaging transducers 22 and therapy transducers 24 that enables imaging in a two-dimensional plane and steering and/or focusing within the two-dimensional plane, in accordance with embodiments of the current disclosure. The energy application device may include a fixed arrangement of therapy transducers 22, imaging transducer 24, and one or more position sensors 40 (e.g., inertial measurement unit (IMU) sensors, optical tracker sensors). The transducers 20 may be automatically steered and/or manually steered (e.g., in real-time) based on image tracking and analysis based on subject ultrasound images and determined therapy zone (e.g., region within the ultrasound image data the therapy transducer is able to deliver therapy to) relative to the desired anatomic target 104 for the subject. In some embodiments, two or more planar arrays in a single unit may be mechanically steered to improve the therapy range of the energy application device. This may facilitate keeping the anatomic target 104 within the therapy zone 102 of the therapy transducers 24, and/or centering the anatomic target 104 within the range of the energy application device to provide effective therapy.

Initially, the alignment controller 30 may receive ultrasound image data (e.g., static image data and/or time-series image data corresponding to a respiration cycle of the subject). Additionally, the alignment controller 30 may receive additional data including the sensor data 40 (e.g., inertial measurement unit (IMU) data, optical tracker data). The image data received by the alignment controller 30 may include two-dimensional plane image data from the fixed imaging transducer 22. The imaging transducer may be located centrally in the probe module 14 and may be connected to two therapy transducers 24 on either end of the imaging transducer 22.

The imaging transducer 24 may generate image data corresponding to a two-dimensional image plane 108. The alignment controller 30 may localize the anatomic target 104 within the received ultrasound image data by comparing the image data to previously acquired image data of the anatomic target 104 and/or previously acquired image data of the anatomical region surrounding the anatomic target 104. The alignment controller 30 may utilize the image data to extract the location of the anatomic target 104 within the ultrasound image data, and may localize the image data relative to the anatomic target 104. It should be understood that the ultrasound image data received by the alignment controller may be fully, partially, or non-inclusive of the anatomic target 104, so in some embodiments the alignment controller 30 may not be able to localize the anatomic target 104 within the image data. The alignment controller 30 may also localize the center point within the anatomic target 104 and/or the volume of the subject that corresponds to the anatomic target 104.

Based on the received image data, the alignment controller 30 may determine an alignment score (e.g., percent alignment score, average percent alignment score, yes and/or no indication of alignment) indicating a level of alignment and/or alignment of the therapy zone 102 with the localized anatomic target 104. The alignment score may be calculated based on determining the therapy zone of the probe module 14 (e.g., region within the ultrasound image data the therapy transducer is able to deliver therapy to) and if the to the anatomic target 104 is within suitable range of the therapy zone 102. The alignment score may also be calculated based on if the anatomic target 104 is within a contour region 110 (e.g., clinician defined subject specific region that is associated with the anatomic target location) to verify reliable alignment and prevent false positives based on inaccurate anatomic target 104 detection by the alignment controller 30. It should be understood, that any suitable criteria may be used to calculate an alignment score using the alignment controller 30 based on the received image data and past subject data and/or clinician defined contour regions 110 within the subject image data. It should be understood, that no external or internal motion is required for two-dimensional tracking, and that the geometry and/or frequency range of each therapy transducer 24 and/or imaging transducer 22 may be optimized for the appropriate imaging and/or therapy needs of the subject.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A neuromodulation delivery system comprising:
an energy application device; and
an alignment controller, wherein the alignment controller is configured to perform acts comprising:
receiving image data from the energy application device, wherein the image data comprises images of internal tissue based on a current position and orientation of the energy application device relative to a subject;
determining an alignment score of the energy application device with respect to an anatomic target based on the image data;
providing a control signal to maintain or change one or both of the current position or orientation of the energy application device in response to the alignment score.

2. The neuromodulation delivery system of claim 1, wherein the energy application device comprises one or more ultrasound imaging transducers and one or more therapy transducers disposed within a housing of the energy application device.

3. The neuromodulation delivery system of claim 2, wherein the position of the energy application device comprises an external position of the energy application device relative to the subject, a position of an imaging transducer, a therapy transducer, or both within the energy application device.

4. The neuromodulation delivery system of claim 1, wherein the anatomic target is extracted from the image data, by localizing the image data relative to the anatomic target, wherein the image data is fully, partially, or non-inclusive of the anatomic target.

5. The neuromodulation delivery system of claim 4, wherein localizing the image data comprises comparing the image data with subject specific data, population specific data, or both.

6. The neuromodulation delivery system of claim 1, wherein the alignment score comprises a cumulative percent alignment score comprising an average of a plurality of alignment scores over time.

7. The neuromodulation delivery system of claim 1, wherein determining the alignment score comprises determining that the anatomic target detected in the image frame is within a therapy zone, wherein the therapy zone comprises a range that the neuromodulation delivery system may deliver therapy to the anatomic target.

8. The neuromodulation delivery system of claim 1, wherein the energy application device comprises a position sensor configured to generate position data over time, and to track position of the energy application device relative to the anatomic target over a subject respiration cycle.

9. The neuromodulation delivery system of claim 1, wherein the control signal is sent to an automation controller within the energy application device, wherein the automation controller electronically adjusts the energy application device position, orientation, or both in response to the control signal.

10. The neuromodulation delivery system of claim 1, wherein the control signal comprises a directional signal indicating a direction to move or orient the energy application device to meet the threshold range.

11. The neuromodulation delivery system of claim 10, wherein the directional signal comprises depth of field (DOF) data and indicates directional movement in a two-dimensional plane relative to the subject.

12. The neuromodulation delivery system of claim 1, wherein the image data comprises two-dimensional plane data, three-dimensional volume data, Doppler data, or any combination thereof.

13. The neuromodulation delivery system of claim 1, wherein the image data comprises time series data.

14. A method comprising:
receiving, via a processor, time-series image data from an energy application device at a current position and orientation relative to a subject, wherein the time-series image data comprises images of internal tissue of the subject at the current position and orientation over time;
determining, via the processor, an alignment score of the energy application device to an anatomic target over time based on the image data;
comparing, via the processor, the alignment score over a time interval corresponding to the time-series image data at the current position and orientation to a predicted alignment score for the time interval at one or both of an additional position or orientation; and
providing a control signal to maintain or change one or both of position or orientation of the energy application device based on the comparison.

15. The method of claim 14, wherein the method further includes receiving time-series image data at an additional time, and determining a predicted therapy dose session time based on the alignment score over time corresponding to the time-series image data at the additional time.

16. The method of claim 14, wherein the time-series image data comprises image data over a respiration cycle of a subject, wherein the time-series image data is used to monitor movement of the internal anatomy of a subject relative to the current position and orientation of the energy application device over the respiration cycle.

17. A tracking system comprising one or more processors and a memory, wherein the one or more processors are configured to execute instructions stored on the memory to perform acts comprising:
receiving image data from an energy application device, wherein the image data comprises images of internal tissue based on a current position and orientation of the energy application device relative to a subject;
receiving target image data comprising an anatomic target and corresponding to internal tissue of the subject;
identifying the anatomic target in an image frame of the image data and tracking the anatomic target in subsequent frames of the image data;
determining an alignment score of the energy application device relative to the anatomic target based on the image data;
comparing the alignment score to a threshold relative to the anatomic target; and
providing a control signal to maintain or change one or both of position or orientation of the energy application device based on the comparison of the alignment score to the threshold.

18. The tracking system of claim 17, wherein the tracking system receives additional data that comprises one or more of respiration cycle data, position of the energy application device over time, or orientation of the energy application device over time.

19. The tracking system of claim 17, wherein the control signal comprises three-dimensional motion data, wherein the three-dimensional motion data indicates movement in one or both of the position or the orientation of the energy application device, an electronic focus of a therapy beam of the energy application device, or both.

20. The tracking system of claim 17, wherein the control signal comprises two-dimensional motion data, wherein the two-dimensional motion data indicates a lateral movement direction of the energy application device, an electronic focus of a therapy beam that can be delivered by the energy application device, or both.
